SEQUENCE LISTING (14 sequences)

SEQ ID NO: 1 - DNA, 17 bp, Artificial Sequence
  Synthetic
ccctccgacgcctgctt

SEQ ID NO: 2 - DNA, 20 bp, Artificial Sequence
  Synthetic
atcatcagcaatgcctcctg

SEQ ID NO: 3 - DNA, 20 bp, Artificial Sequence
  Synthetic
ggaacaaggatgagatcagc

SEQ ID NO: 4 - DNA, 19 bp, Artificial Sequence
  Synthetic
ctggccgttgaagtagagg

SEQ ID NO: 5 - DNA, 25 bp, Artificial Sequence
  Synthetic
gggaaatcgtgcgtgacattaagga

SEQ ID NO: 6 - DNA, 23 bp, Artificial Sequence
  Synthetic
tgtgttggcgtacaggtctttgc

SEQ ID NO: 7 - DNA, 17 bp, Artificial Sequence
  Synthetic
ccctccgacgcctgctt

SEQ ID NO: 8 - DNA, 20 bp, Artificial Sequence
  Synthetic
atcatcagcaatgcctcctg

SEQ ID NO: 9 - DNA, 24 bp, Artificial Sequence
  Synthetic
aagtaccccgagaacttcttcctg

SEQ ID NO: 10 - DNA, 22 bp, Artificial Sequence
  Synthetic
tgaggtccaagtcgtgcttgtg

SEQ ID NO: 11 - DNA, 19 bp, Artificial Sequence
  Synthetic
tccacaaatcagacggcag

SEQ ID NO: 12 - DNA, 20 bp, Artificial Sequence
  Synthetic
cataaggggcagcaatgaac

SEQ ID NO: 13 - DNA, 23 bp, Artificial Sequence
  Synthetic
tgtgttggcgtacaggtctttgc

SEQ ID NO: 14 - DNA, 20 bp, Artificial Sequence
  Synthetic
atcatcagcaatgcctcctg

(19) United States
(12) Patent Application Publication
(10) Pub. No.: US 2004/0215087 A1
(43) Pub. Date: Oct. 28, 2004

(54) Compositions and methods for the detection of biomarkers associated with cardiovascular disease

(76) Inventors: Mario Hugo Genero, Pilar (AR); Elizabeth Edit Ojeda, Pilar (AR)

Correspondence Address: Peter G Carroll, Medlen & Carroll, Suite 350, 101 Howard Street, San Francisco, CA, 94105, US

(21) Appl. No.: 10/474,982

(22) PCT Filed: Jun. 5, 2001
(86) PCT No.: PCT/GB01/02464

(51) Int. Cl. A61B 5/02

(52) U.S. Cl. 600/508

(57) ABSTRACT

Compositions and methods of detecting biomarkers associated with cardiovascular disease through the use of cardiac tissue microarrays are provided. The compositions of the present invention comprise a human cardiovascular tissue microarray comprising a plurality of tissues derived from at least one human donor, and reflecting at least one state of cardiovascular disease. The methods of the present invention comprise treating a human cardiovascular tissue microarray under conditions such that a biomarker associated with cardiovascular disease is detected.

### FIELD OF THE INVENTION

The present invention relates generally to the field of cardiovascular disease, and in particular, to compositions and enhanced methods of detecting biomarkers associated with cardiovascular disease through the use of cardiac tissue microarrays.

### BACKGROUND

Cardiovascular disease is a debilitating illness that afflicts millions of people in the world each year. Indeed, in 1997, over 450,000 people in the U.S. alone died from myocardial infarctions; one of every five deaths in that calendar year. In addition to myocardial infarction (heart attack), cardiovascular disease results in hypertension, angina, arteriosclerosis, and atherosclerosis. Angina, for example, accounts for more than 1 million hospital admissions annually in the U.S., and 6-8 percent of patients with this condition either have non-fatal myocardial infarction, or die, within the first year after diagnosis.

Currently, physicians are able to diagnose cardiovascular disease in patients who have already begun to experience symptoms. For example, the levels of certain cardiac-associated enzymes, such as creatine kinase, are elevated after myocardial infarction, and may be detected an enzyme-specific assay. However, there is no effective means of pre-symptomatic diagnosis of cardiovascular disease, let alone for detecting the underlying genetic risks for cardiovascular disease. Thus, what is needed is the development of tools and methods that will allow the pre-symptomatic identification of cardiovascular disease, and the detection of the associated genetic risk factors.

### SUMMARY OF THE INVENTION

The present invention relates generally to the field of cardiovascular disease, and in particular, to devices comprising cardiac tissue microarrays useful for the detection of biomarkers associated with cardiovascular disease. Specifically, the device of the present invention provides tools comprising a cardiac tissue microarray that will allow the pre-symptomatic identification of cardiovascular disease, and the associated genetic risk factors.

In one embodiment, the present invention contemplates a device for the detection of biomarkers associated with cardiovascular disease, comprising a tissue microarray, wherein said microarray comprises a plurality of human cardiovascular tissue samples placed on the surface of said microarray. In another embodiment, the present invention contemplates a device having a substantially flat surface, wherein a plurality of human cardiovascular tissue samples from at least one human donor are arrayed on said surface, wherein said plurality comprises a first sample from a first disease state and a second tissue sample from a second disease state.

It is not intended that the device of the present invention be limited to any specific source of cardiovascular tissue. In one embodiment, said tissue is derived from a human source. In another embodiment, said tissue is derived from a non-human animal source.

It is not intended that the device of the present invention be limited to any specific type of cardiovascular tissue samples. In one embodiment, said tissue samples are selected from the group of tissues comprising different sources of venous and arterial vessels (e.g. aorta, coronary, saphen, etc.), ventricles, and auricles.

It is not intended that the tissue samples of the device of the present invention be limited to diseased cardiovascular tissue. In one embodiment, said tissue is diseased. In another embodiment, said tissue is non-diseased, and serves as a negative control for the detection of cardiovascular disease biomarkers. In a preferred embodiment, the present invention contemplates a microarray comprising both diseased and non-diseased cardiovascular tissue samples. In an alternative embodiment, the present invention contemplates a microarray comprising both diseased and non-diseased cardiovascular tissue samples, wherein said diseased samples represent at least two different stages of disease (e.g. chronologically) or disease states associated with the progression of cardiovascular disease. In another embodiment, a device having a substantially flat surface, wherein a plurality of human cardiovascular tissue samples from at least one human donor are arrayed on said surface, wherein said plurality comprises a first sample from a first disease state and a second tissue sample from a second disease state, and wherein said second disease state is a later (“in time”) disease state than said first disease state.

It is not intended that the diseased tissue contemplated by the present invention be limited to any specific cardiovascular disease state. In one embodiment, said disease state is selected from the group of stable angina, unstable angina, non-Q-wave myocardial infarction, and Q-wave myocardial infarction.

It is not intended that the tissue samples of the device of the present invention be limited to being derived from a single source. In one embodiment, said cardiovascular tissue samples on the microarray are derived from one human source. In another embodiment, said cardiovascular tissue samples on the microarray are derived from more than one human source. Moreover, it is not intended that the tissue samples of the device of the present invention be limited to living donor specimens. In one embodiment, said cardiovascular tissue samples comprise cadaveric donor specimens. In another embodiment, said cardiovascular tissue samples comprise living donor specimens including, but not limited to, biopsy specimens.

It is not intended that the device of the present invention be limited to any specific number of cardiovascular tissue samples. In one embodiment, the present invention contemplates a microarray comprising 4-16 tissue samples. In another embodiment, the present invention contemplates a microarray comprising 16-42 tissue samples. In yet another embodiment, the present invention contemplates a microarray comprising 42-98 tissue samples. In still other embodiments, more than 98 tissue samples are arrayed.

The present invention also contemplates a cardiac tissue microarray comprising cardiovascular tissues which are derived from different regions of the heart (i.e. a topographical microarray). (See e.g., FIG. 8). For example, in one embodiment, the present invention contemplates a device having a substantially flat surface, wherein a plurality of human cardiovascular tissue samples from at least one donor are arrayed on said surface, wherein said plurality comprises a first sample from one region of the heart and a second sample from a second region of the heart.

In another embodiment, the present invention contemplates a cardiac tissue microarray comprising cardiovascular tissues that are representative of the various stages in the progression of cardiovascular disease (i.e. a chronological microarray). For example, in one embodiment, the present invention contemplates a device having a substantially flat surface, wherein a plurality of human cardiovascular tissue samples from at least one donor are arrayed on said surface, wherein said plurality comprises tissue from a donor suffering from a cardiovascular disease selected from the group consisting of stable angina, unstable angina, non-Q-wave myocardial infarction, and Q-wave myocardial infarction. In another embodiment, said plurality further comprises non-diseased cardiovascular tissues.

In an alternative embodiment, the present invention also contemplates a cardiac tissue microarray comprising cardiovascular tissues which are derived from different regions of the heart, and which are representative of the various stages in the progression of cardiovascular disease (i.e. a topological and chronological microarray). For example, in one embodiment, the present invention contemplates a device having a substantially flat surface, wherein a plurality of human cardiovascular tissue samples from at least one donor are arrayed on said surface, wherein said plurality comprises a first sample from one region of the heart and a second sample from a second (and different) region of the heart, and wherein said plurality comprises tissue from a donor suffering from a cardiovascular disease selected from the group consisting of stable angina, unstable angina, non-Q-wave myocardial infarction, and Q-wave myocardial infarction. In another embodiment, said plurality further comprises non-diseased cardiovascular tissues.

It is not intended that the device of the present invention be limited to cardiovascular tissue samples derived from any specific region of the heart. In one embodiment, said tissue samples are derived from a region of the heart selected from the group consisting of aorta, right atrium, right ventricle, circumflex, septal wall, obtuse marginal branch of the left coronary artery, anterior descending branch of the left coronary artery, left atrium, left ventricle, sinuatrial nodal (sinus node) artery, conus arteriosus branch of the right coronary artery, right coronary artery, acute marginal branch of the right coronary artery, atrioventricular (A.V.) nodal artery, and posterior descending branch of the right coronary artery. (See e.g., FIGS. 8 & 9).

The present invention also particularly relates to an enhanced method of detecting biomarkers associated with cardiovascular disease through the use of cardiac tissue microarrays. The methods of the present invention comprise the use of said cardiac tissue microarray such that a plurality of cardiovascular tissues can be rapidly screened for the presence or absence of biomarkers associated (or suspected of being associated) with cardiovascular disease.

In one embodiment, the present invention contemplates a method for the detection of biomarkers associated with cardiovascular disease, comprising: a) providing the cardiac tissue microarray of the present invention; and b) subjecting said microarray to analysis by a method selected from the group of histological analysis, immunological analysis, nucleic acid hybridization analysis, and combinations thereof, such that the presence or absence of a biomarker associated with cardiovascular disease is determined.

It is not intended that the method of the present invention be limited to any specific means of histological analysis to determine the presence or absence of a biomarker associated (or suspected of being associated) with cardiovascular disease. In one embodiment, said histological analysis comprises hematoxylin and eosin staining. In another embodiment, said histological analysis is selected from the group of light microscopy, phase-contrast microscopy, and osmium tetroxide/glutaraldehyde treatment followed by electron microscopy.

It is not intended that the method of the present invention be limited to any specific means of immunological analysis to determine the presence or absence of a biomarker associated with cardiovascular disease. In one embodiment, said immunological analysis comprises immunohistochemistry.

It is not intended that the method of the present invention be limited to any specific means of nucleic acid hybridization analysis to determine the presence or absence of a biomarker associated with cardiovascular disease. In one embodiment, said nucleic acid hybridization analysis comprises in situ reverse transcriptase polymerase chain reaction (IS RT-PCR). In another embodiment, said nucleic acid hybridization analysis comprises fluorescent in situ hybridization (FISH).

Liver Disease

It is not intended that the present invention be limited to devices comprising cardiovascular tissue microarrays. In an alternative embodiment, the present invention contemplates a device for the detection of biomarkers associated with liver disease, comprising a tissue microarray, wherein said microarray comprises a plurality of human liver tissue samples placed on the surface of said microarray.

It is not intended that the device of the present invention be limited to any species-specific source of liver tissue. In one embodiment, said tissue is derived from a human source. In another embodiment, said tissue is derived from a non-human source.

It is not intended that the liver tissue of the device of the present invention be limited to any specific liver cell type. In one embodiment, said tissue samples are comprised of liver cell types selected from the group of parenchymal (hepatic) cells, cells associated with the walls of the hepatic sinusoids, or blood cells in the lumina of the hepatic sinusoids.

It is not intended that the tissue samples of the device of the present invention be limited to diseased liver tissue. In one embodiment, said tissue is diseased. In another embodiment, said tissue is non-diseased, and serves as a negative control for the detection of liver disease biomarkers. In a preferred embodiment, the present invention contemplates a microarray comprising both diseased and non-diseased liver tissue samples. In an alternative embodiment, the present invention contemplates a microarray comprising both diseased and non-diseased liver tissue samples, wherein said diseased samples represent the different stages of disease (e.g. chronologically) or disease states associated with the progression of liver disease.

It is not intended that the diseased tissue contemplated by the present invention be limited to any specific liver disease state. In one embodiment, said disease state is selected from the group of hepatitis, fibrosis, hepatocellular cancer, and cirrhosis of the liver.

It is not intended that the tissue samples of the device of the present invention be limited to being derived from a single source. In one embodiment, said liver tissue samples on the microarray are derived from one human source. In another embodiment, said liver tissue samples on the microarray are derived from more than one human source. Moreover, it is not intended that the tissue samples of the device of the present invention be limited to living donor specimens. In one embodiment, said liver tissue samples on the microarray comprise cadaveric donor specimens. In another embodiment, said liver tissue samples on the microarray comprise living donor specimens including, but not limited to, biopsy specimens.

It is not intended that the device of the present invention be limited to any specific number of liver tissue samples. In one embodiment, the present invention contemplates a microarray comprising 4-16 tissue samples. In another embodiment, the present invention contemplates a microarray comprising 16-42 tissue samples. In yet another embodiment, the present invention contemplates a microarray comprising 42-98 tissue samples. In still other embodiments, more than 98 tissue samples are arrayed.

The present invention also particularly relates to an enhanced method of detecting biomarkers associated with liver disease through the use of liver tissue microarrays. The methods of the present invention comprise the use of said liver tissue microarray such that a plurality of liver tissues can be rapidly screened for the presence or absence of numerous biomarkers associated with liver disease.

In one embodiment, the present invention contemplates a method for the detection of biomarkers associated with liver, comprising: a) providing the liver tissue microarray of the present invention; and b) subjecting said microarray to analysis by a method selected from the group of histological analysis, immunological analysis, nucleic acid hybridization analysis, and combinations thereof, such that the presence or absence of a biomarker associated with liver disease is determined.

It is not intended that the method of the present invention be limited to any specific means of histological analysis to determine the presence or absence of a biomarker associated with liver disease. In one embodiment, said histological analysis comprises hematoxylin and eosin staining. In another embodiment, said histological analysis is selected from the group of light microscopy, phase-contrast microscopy, and osmium tetroxide/glutaraldehyde treatment followed by electron microscopy.

It is not intended that the method of the present invention be limited to any specific means of immunological analysis to determine the presence or absence of a biomarker associated with liver disease. In one embodiment, said immunological analysis comprises immunohistochemistry.

It is not intended that the method of the present invention be limited to any specific means of nucleic acid hybridization analysis to determine the presence or absence of a biomarker associated with liver disease. In one embodiment, said nucleic acid hybridization analysis comprises in situ reverse transcriptase polymerase chain reaction (RT-PCR). In another embodiment, said nucleic acid hybridization analysis comprises fluorescent in situ hybridization (FISH).

Kidney Disease

It is not intended that the present invention be limited to devices comprising liver tissue microarrays. In a further alternative embodiment, the present invention contemplates a device for the detection of biomarkers associated with kidney disease, comprising a tissue microarray, wherein said microarray comprises a plurality of human kidney tissue samples placed on the surface of said microarray.

It is not intended that the device of the present invention be limited to any species-specific source of kidney tissue. In one embodiment, said tissue is derived from a human source. In another embodiment, said tissue is derived from a non-human source.

It is not intended that the tissue samples of the device of the present invention be limited to diseased kidney tissue. In one embodiment, said tissue is diseased. In another embodiment, said tissue is non-diseased, and serves as a negative control for the detection of kidney disease biomarkers. In a preferred embodiment, the present invention contemplates a microarray comprising both diseased and non-diseased kidney tissue samples. In an alternative embodiment, the present invention contemplates a microarray comprising both diseased and non-diseased kidney tissue samples, wherein said diseased samples represent the different stages of disease (e.g. chronologically) or disease states associated with the progression of kidney disease.

It is not intended that the diseased tissue contemplated by the present invention be limited to any specific kidney disease state. In one embodiment, said disease state is selected from the group of idiopathic membranoproliferative glomerulonephritis (MPGN), Alport Syndrome, and Goodpasture's Syndrome.

It is not intended that the tissue samples of the device of the present invention be limited to being derived from a single source. In one embodiment, said kidney tissue samples on the microarray are derived from one human source. In another embodiment, said kidney tissue samples on the microarray are derived from more than one human source. Moreover, it is not intended that the tissue samples of the device of the present invention be limited to living donor specimens. In one embodiment, said kidney tissue samples on the microarray comprise cadaveric donor specimens. In another embodiment, said kidney tissue samples on the microarray comprise living donor specimens including, but not limited to, biopsy specimens.

It is not intended that the device of the present invention be limited to any specific number of kidney tissue samples. In one embodiment, the present invention contemplates a microarray comprising 4-16 tissue samples. In another embodiment, the present invention contemplates a microarray comprising 16-42 tissue samples. In yet another embodiment, the present invention contemplates a microarray comprising 42-98 tissue samples. In still other embodiments, more than 98 tissue samples are arrayed.

The present invention also particularly relates to an enhanced method of detecting biomarkers associated with kidney disease through the use of kidney tissue microarrays. The methods of the present invention comprise the use of said kidney tissue microarray such that a plurality of kidney tissues can be rapidly screened for the presence or absence of numerous biomarkers associated with kidney disease.

In one embodiment, the present invention contemplates a method for the detection of biomarkers associated with kidney disease, comprising: a) providing the kidney tissue microarray of the present invention; and b) subjecting said microarray to analysis by a method selected from the group of histological analysis, immunological analysis, nucleic acid hybridization analysis, and combinations thereof, such that the presence or absence of a biomarker associated with kidney disease is determined.

It is not intended that the method of the present invention be limited to any specific means of histological analysis to determine the presence or absence of a biomarker associated with kidney disease. In one embodiment, said histological analysis comprises hematoxylin and eosin staining. In another embodiment, said histological analysis is selected from the group of light microscopy, phase-contrast microscopy, and osmium tetroxide/glutaraldehyde treatment followed by electron microscopy.

It is not intended that the method of the present invention be limited to any specific means of immunological analysis to determine the presence or absence of a biomarker associated with kidney disease. In one embodiment, said immunological analysis comprises immunohistochemistry.

It is not intended that the method of the present invention be limited to any specific means of nucleic acid hybridization analysis to determine the presence or absence of a biomarker associated with kidney disease. In one embodiment, said nucleic acid hybridization analysis comprises in situ reverse transcriptase polymerase chain reaction (RT-PCR). In another embodiment, said nucleic acid hybridization analysis comprises fluorescent ill situ hybridization (FISH).

Brain Disease

It is not intended that the present invention be limited to devices comprising kidney tissue microarrays. In a further alternative embodiment, the present invention contemplates a device for the detection of biomarkers associated with brain disease comprising a tissue microarray, wherein said microarray comprises a plurality of human brain tissue samples placed on the surface of said microarray.

It is not intended that the device of the present invention be limited to any species-specific source of brain tissue. In one embodiment, said tissue is derived from a human source. In another embodiment, said tissue is derived from a non-human source.

It is not intended that the tissue samples of the device of the present invention be limited to diseased brain tissue. In one embodiment, said tissue is diseased. In another embodiment, said tissue is non-diseased, and serves as a negative control for the detection of brain disease biomarkers. In a preferred embodiment, the present invention contemplates a microarray comprising both diseased and non-diseased brain tissue samples. In an alternative embodiment, the present invention contemplates a microarray comprising both diseased and non-diseased brain tissue samples, wherein said diseased samples represent the different stages of disease (e.g. chronologically) or disease states associated with the progression of brain disease.

It is not intended that the diseased tissue contemplated by the present invention be limited to any specific brain disease state. In one embodiment, said disease state is selected from the group of Creutzfeldt-Jakob Disease (CJD) and Transmissible Spongiform Encephalopathy (TSE).

It is not intended that the tissue samples of the device of the present invention be limited to being derived from a single source. In one embodiment, said brain tissue samples on the microarray are derived from one human source. In another embodiment, said brain tissue samples on the microarray are derived from more than one human source. Moreover, it is not intended that the tissue samples of the device of the present invention be limited to living donor specimens. In one embodiment, said brain tissue samples on the microarray comprise cadaveric donor specimens. In another embodiment, said brain tissue samples on the microarray comprise living, non-human, donor specimens including, but not limited to, biopsy specimens.

It is not intended that the device of the present invention be limited to any specific number of brain tissue samples. In one embodiment, the present invention contemplates a microarray comprising 4-16 tissue samples. In another embodiment, the present invention contemplates a microarray comprising 1642 tissue samples. In yet another embodiment, the present invention contemplates a microarray comprising 42-98 tissue samples. In still other embodiments, more than 98 tissue samples are arrayed.

The present invention also particularly relates to an enhanced method of detecting biomarkers associated with brain disease through the use of brain tissue microarrays. The methods of the present invention comprise the use of said brain tissue microarray such that a plurality of brain tissues can be rapidly screened for the presence or absence of numerous biomarkers associated with brain disease.

In one embodiment, the present invention contemplates a method for the detection of biomarkers associated with brain disease, comprising: a) providing the brain tissue microarray of the present invention; and b) subjecting said microarray to analysis by a method selected from the group of histological analysis, immunological analysis, nucleic acid hybridization analysis, and combinations thereof, such that the presence or absence of a biomarker associated with brain disease is determined.

It is not intended that the method of the present invention be limited to any specific means of histological analysis to determine the presence or absence of a biomarker associated with brain disease. In one embodiment, said histological analysis comprises hematoxylin and eosin staining. In another embodiment, said histological analysis is selected from the group of light microscopy, phase-contrast microscopy, and osmium tetroxidelglutaraldehyde treatment followed by electron microscopy.

It is not intended that the method of the present invention be limited to any specific means of immunological analysis to determine the presence or absence of a biomarker associated with brain disease. In one embodiment, said immunological analysis comprises immunohistochemistry. In another embodiment, said immunological analysis comprises Fluorescence Correlation Spectroscopy (FCS) as noted in Geise et al., “Putting prions into focus: application of single molecule detection to the diagnosis of prion diseases,” Arch. Virol. Suppl., (16): 161-71 (2000).

It is not intended that the method of the present invention be limited to any specific means of nucleic acid hybridization analysis to determine the presence or absence of a biomarker associated with brain disease. In one embodiment, said nucleic acid hybridization analysis comprises in situ reverse transcriptase polymerase chain reaction (RT-PCR). In another embodiment, said nucleic acid hybridization analysis comprises fluorescent in situ hybridization (FISH).

### DESCRIPTION OF THE FIGURES

To facilitate an understanding of the invention, a number of figures are included herein.

FIG. 1 depicts a microarray comprised of cardiovascular tissue derived from a single donor suffering from cardiovascular disease. In this figure, tissue samples are arranged in duplicate rows as indicated by “[duplicate]” or “[d].”

FIG. 2 depicts a microarray comprised of cardiovascular tissue derived from two donors suffering from cardiovascular disease. In this figure, tissue samples are arranged in duplicate rows as indicated by “[duplicate]” or “[d].”

FIG. 3 depicts a microarray comprised of cardiovascular tissue derived from three donors suffering from cardiovascular disease. In this figure, tissue samples are arranged in duplicate rows as indicated by “[duplicate]” or “[d].”

FIG. 4 depicts a microarray comprised of cardiovascular tissue derived from four donors suffering from cardiovascular disease. In this figure, tissue samples are arranged in duplicate rows as indicated by “[duplicate]” or “[d].”

FIG. 5 depicts a microarray comprised of cardiovascular tissue derived from five donors suffering from cardiovascular disease, and from one non-diseased (cardiovascular) donor. In this figure, tissue samples are arranged in duplicate rows as indicated by “[duplicate]” or “[d].”

FIG. 6 is a flowchart that depicts One approach for the utilization of the methods and compositions of the present invention to detect the presence or absence of biomarkers associated with cardiovascular disease.

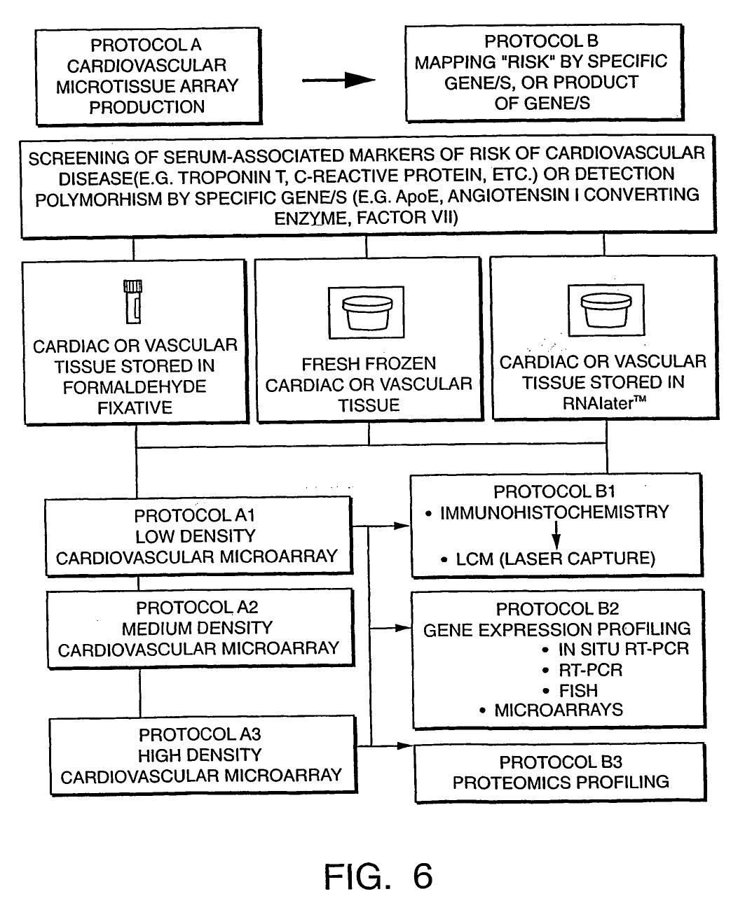

FIG. 7 depicts the general progression of acute coronary syndromes beginning with ischemic discomfort, and terminating in either unstable angina, non-Q-wave myocardial infarction, or Q-wave myocardial infarction.

FIG. 8 depicts a topological map of the coronary arterial tree as viewed in one of the projections commonly used in coronary arteriography. The map divides the human heart into discrete sections (e.g. A-1, B-2, C-3, etc.) which correspond with identically numbered sections of functional and topological arterial tissue microarrays as described herein. (See also FIGS. 9-14).

FIG. 9 depicts one embodiment of a functional and topological arterial tissue microarray (as described herein) in which three distinct atherosclerotic cardiovascular disease states (i.e. coronary heart disease, stroke, and peripheral arterial vascular disease) are represented. The sections labeled A-1 through A-3, B-1 through B-3, & C-1 through C-3 correspond to the sections depicted in FIG. 8 and represent coronary arterial tissues affected by coronary heart disease (CHD). The sections labeled D-1 through D-3 correspond to arterial tissues that are implicated in stroke. The sections labeled E-1 through E-3 correspond to arterial tissues that are implicated in peripheral arterial vascular disease (PVD). (See also FIGS. 8, 10-14).

FIG. 10 depicts one embodiment of a functional and topological arterial tissue microarray (lower portion) wherein the sections labeled A-1 through A-3, B-1 through B-3, & C-1 through C-3 (upper portion) correspond to the sections depicted in FIG. 8 and represent coronary arterial tissues affected by coronary heart disease (CHD). This particular embodiment of the microarray depicts the aortic tissue samples placed in section A-1 of said microarray.

FIG. 11 depicts one embodiment of a functional and topological arterial tissue microarray (lower portion) with sections labeled as described in the figure legends of FIGS. 8 & 9. This particular embodiment of the microarray depicts the internal carotid tissue samples placed in section D-1 of said microarray.

FIG. 12 depicts one embodiment of a functional and topological arterial tissue microarray (lower portion) with sections labeled as described in the figure legends of FIGS. 8 & 9. This particular embodiment of the microarray depicts the abdominal aorta tissue samples placed in section E-1 of said microarray.

FIG. 13 depicts one embodiment of a functional and topological arterial tissue microarray (lower portion) with sections labeled as described in the figure legends of FIGS. 8 & 9. This particular embodiment of the microarray depicts the tissue samples (e.g. common femoral artery, deep femoral artery, and superior femoral artery) placed in section E-2 of said microarray.

FIG. 14 depicts one embodiment of a functional and topological arterial tissue microarray (lower portion) with sections labeled as described in the figure legends of FIGS. 8 & 9. This particular embodiment of the microarray depicts the tissue samples (e.g. tibial artery, popliteal artery, and peroneal artery) placed in section E-3 of said microarray.

FIG. 15 depicts one embodiment of the device of the present invention comprising a high-density cardiovascular tissue microarray (e.g. a cardiovascular tissue microarray having 432 samples). Section 100 indicates the device itself, whereas Sections 101 and 102 respectively indicate the surface of the device, and the cardiovascular tissue samples placed thereupon.

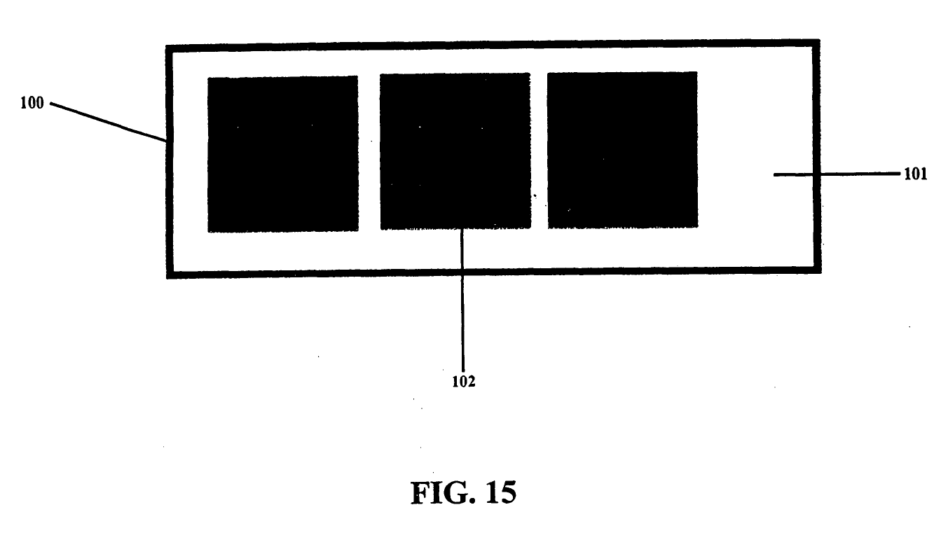

### DEFINITIONS

“Alport syndrome,” as used herein, refers to an inherited progressive kidney disease with an estimated gene frequency of 1:5000. The disease is characterized by hematuria and terminal renal failure, often accompanied by familial hearing loss and ocular lesions such as lenticonus. It is usually inherited as an X chromosome-linked dominant trait, but autosomal forms have also been described.

“Angina” or “Angina Pectoris,” as used herein, refers to chest pain that is caused by blockages in the arteries that supply blood to the heart. Angina is further sub-divided and classified by the length of time between each onset of chest pain as follows:

1. acute angina—while at rest (within the 48 hours before presentation),

2. sub-acute angina—while at rest (within the previous month but not within the 48 hours before presentation), or

3. new onset of accelerated (progressively more severe) angina;

“Acute coronary syndrome,” or “ACS,” as used herein, refers to the spectrum of conditions including, but not limited to, unstable angina (UA), non-Q-wave myocardial infarction (which generally presents without ST-segment elevation), and Q-wave myocardial infarction (which generally presents with ST-segment elevation). UA and non-ST-segment elevation myocardial infarction (NSTEMI) are acute coronary syndromes (ACSs) that are characterized by an imbalance between myocardial oxygen supply and demand. The most common cause is reduced myocardial perfusion that results from coronary artery narrowing caused by a non-occlusive thrombus that has developed on a disrupted atherosclerotic plaque. Abnormal constriction of the coronary arteries may also be responsible. UA and NSTEMI are considered to be closely related conditions whose pathogenesis and clinical presentations are similar but of differing severity (i.e., they differ primarily in whether the ischemia is severe enough to cause sufficient myocardial damage to release detectable quantities of a marker of myocardial injury, most commonly, troponin I [TnI], troponin T [TnT], or the MB isoenzyme of creatine phosphokinase [CK-MB]). Once it has been established that no biochemical marker of myocardial necrosis has been released, the patient with an ACS may be considered to have experienced UA, whereas the diagnosis of NSTEMI is established if a marker of myocardial injury has been released.

“Atherothrombotic cardiovascular disease,” as used herein, refers to a diffuse atherosclerotic condition involving the heart (coronary arteries), brain (carotid, vertebral, and cerebral arteries), and peripheral arteries. Indeed, most of the risk factors that apply to one arterial bed also apply to the others. It is, therefore, not surprising that the presence of one atherosclerotic cardiovascular disease increases the risk of developing other such diseases.

“Biomarker,” as used herein, refers to any biologically-based marker (e.g. gene, gene fragment, gene product, nucleic acid, protein, protein fragment, peptide, polypeptide, or epitope), that the presence, absence, or variation in expression of, is associated with a particular disease state. The term “gene” encompasses both cDNA and genomic forms of a given gene. The present invention contemplates devices and methods for the detection of biomarkers associated with cardiovascular,kidney, liver, and brain diseases. Specifically, the present invention contemplates cardiovascular tissue microarrays for the detection of biomarkers associated with cardiovascular disease such as the PAI-1 geneand gene product.

“Cardiovascular disease,” as used herein, refers to any disease which affects the cardiovascular system including, but not limited to, thrombophilia, atherosclerosis, and arteriosclerosis.

“Goodpasture's syndrome,” as used herein, refers to the association of severe nephritis with pulmonary hemorrhage, and although various immunopathological mechanisms may underlie this clinical picture, the eponym is not generally reserved for those cases due to auto-antibodies to the glomerular basement membrane (GBM).

“Hybridization,” as used herein, refers to the formation of sequence-specific, base-paired duplexes from any combination of nucleic acid fragments. Hybridization, regardless of the method used, requires some complementarity between the sequence of interest (the target sequence) and the fragment of nucleic acid used to detect the target sequence and/or perform the test (e.g., the probe). Thus, these duplexes may be completely complementary or may include mismatched sequences. For example, where it is desired to detect simply the presence or absence of DNA or RNA, it is only important that the hybridization method ensures hybridization when the relevant sequence is present; conditions can be selected where both partially complementary probes and completely complementary probes will hybridize. However, other diagnostic applications may require that the method of hybridization distinguish between variant target sequences. For example, it may be of interest that a particular allelic variant is present. Methods have been devised to enable discrimination between partial and complete complementarity. One approach is to take advantage of the temperature requirements of the specific hybridization under study. In typical melting curve experiments, such as those described by Wallace et al., Nucl. Acids Res., 6: 35632 (1979) and Nucl. Acids Res. 9: 879 (1981), it is observed that partially complementary probe-target duplexes display a lower thermal stability than do completely complementary probe-target duplexes. The best estimate is that a 1% mismatch causes a reduction in the thermal stability of duplexes, as measured by the duplex melting temperature (Tₘ), by 1° C. See R. J. Britten and E. H. Davidson, in: Nucleic Acid Hybridisation, (B. D. Hames and S. J. Higgins, eds.) (IRL Press, Washington, 1985)(pp. 3-15). The Tₘ is also affected by the length of the base-paired region of a duplex, according to the equation D=500/L, wherein D is the reduction in Tₘ (° C.) and L is the length of the base-paired duplex. The base composition of the duplex is another factor which affects its stability. In normal salt solutions, GC base pairs are more stable than AT pairs, thus the Tₘ of a particular duplex is related to its GC content according to the equation Tₘ=0.41(% GC)+69.3.

“Tissue microarray,” as used herein, refers to an orderly arrangement of a plurality of tissue samples or specimens, said samples or specimens ranging in size of up to approximately 1000 microns in diameter (more preferably 200-400 microns in diameter, even more preferably 401-500 microns in diameter, and still more preferably 501-600 microns in diameter) placed on the surface of a solid support (e.g. a microscope slide). A “single-source tissue microarray” refers to a tissue microarray wherein the tissue samples are derived from a single source (e.g. a single cadaveric donor). A “comparative tissue microarray” refers to a tissue microarray wherein the tissues samples 1) are derived from more than one source (e.g. multiple cadaveric donors), 2) are comprised of either different tissue types (e.g. aorta, circumflex, venous, arterial, or cardiac tissue) or different sections of the same tissue type, or 3) are compared to non-diseased control tissues specimens similarly placed on the surface of the microarray. A “chronological tissue microarray” refers to a tissue microarray wherein the tissue samples are arranged such as to reflect the chronological progression of a disease (e.g. following the progression stable angina, unstable angina, non-Q-wave myocardial infarction, and Q-wave myocardial infarction).

The present invention contemplates a “high density” tissue microarray, which refers to a tissue microarray in which up to approximately 1200 tissue samples are arrayed in a 40 mm 5×25 mm recipient array block (e.g. paraffin block), and placed on the surface of a 3 in.×1 in. (or 75 mm×25 mm) microscope slide. The present invention also contemplates a “medium-high density” tissue microarray, which refers to a tissue microarray in which up to approximately 700 tissue samples are arrayed in a 40 mm×25 mm recipient array block (e.g. paraffin block), and placed on the surface of a 3 in.×1 in. (or 75 mm×25 mm) microscope slide. The present invention further contemplates a “medium density” tissue microarray, which refers to a tissue microarray in which up to approximately 300-500 tissue samples are arrayed in a 40 mm×25 mm recipient array block (e.g. paraffin block), and placed on the surface of a 3 in.×1 in. (or 75 mm×25 mm) microscope slide. Finally, the present invention contemplates a “low density” tissue microarray which refers to a tissue microarray in which up to approximately 100-200 tissue samples are arrayed in a 40 mm×25 mm recipient array block (e.g. paraffin block), and placed on the surface of a 3 in.×1 in. (or 75 mm×25 mm) microscope slide. Although tissue samples are arrayed in 40 mm×25 mm recipient array blocks, the present invention contemplates tissue microarrays comprising multiple recipient array blocks (e.g. three low density tissue microarray recipient blocks placed together on the surface of a 3 in.×1 in. microscope slide with a total number of 300-600 samples present on the slide).

“Placed on the surface,” as used herein, refers to the process by which tissue specimens are positioned and contacted with, linked to, adhered to, attached to, bound to, or affixed to a (usually flat) surface suitable for mounting tissue (e.g. glass, plastic, silicon, metal, gel, etc.). The term encompasses the covalent, non-covalent, and hydrogen bonding of tissue specimens to the surface to create a tissue microarray. A convenient surface is that of a conventional glass microscope slide.

“Plurality of cardiovascular tissue(s),” as used herein, refers to a number of cardiovascular tissue specimens to be placed on the surface of a cardiovascular tissue microarray wherein the number is preferably at least three tissue specimens, more preferably, 3-16 tissue specimens, even more preferably, 16-42 tissue specimens, and still more preferably, 42-98 tissue specimens, and most preferably, greater than 98 tissue specimens, placed upon the surface of a 3 in.×1 in. (or 75 mm×25 mm) microscope slide. For example, the present invention contemplates cardiovascular tissue microarrays having up to approximately 432 tissue specimens, as well as, cardiovascular tissue microarrays having up to approximately 864 tissue specimens, placed upon the surface of a 3 in.×1 in. (or 75 mm×25 mm) microscope slide.

“Substantially flat surface,” as used herein, refers to surfaces useful for the construction of tissue microarrays as contemplated by the present invention. Said term encompasses surfaces which are completely flat, partially flat, and partially curved. For example, in one embodiment, the present invention contemplates a cardiovascular tissue microarray comprising a standard 3 in.×1 in. (or 75 mm×25 mm) microscope slide having a completely flat surface (i.e. a surface having an angle equal to zero or 180°). In another embodiment, said microscope slide has a partially flat surface (i.e. a surface having an angle between 0-45° or 135-180°). In another embodiment, said microscope slide has a partially curved surface (i.e. a surface having an angle between 45-90° or 90-135°). In a further embodiment, the present invention contemplates a cardiovascular tissue microarray comprising a circular wheel upon which tissue samples are placed, wherein any discrete point on the surface of said wheel is completely flat, partially flat, or partially curved. The term “Substantially flat surface,” also encompasses surfaces which have microirregularities (e.g. surfaces having ridges or grooves that are (only) visible through the aid of a microscope).

### DETAILED DESCRIPTION OF THE INVENTION

Certain genetic markers, or “biomarkers,” associated with cardiovascular disease have been identified. For example, the expression of the gene product of the plasminogen activator inhibitor type 1 (PAI-1) gene has been linked to cardiovascular diseases (See Kohler, H. P. & Grant, P. J., “Plasminogen-activator inhibitor type 1 and coronary artery disease,” New Eng. J. Med., 342: 1792-1801 (2000)) such as, for example, thrombophilia. (See Engesser et al., “Elevated plasminogen activator inhibitor (PAI), a cause of thrombophilia? A study in 203 patients with familial or sporadic venous thrombophilia,” Thromb. Haentost., 62: 673-680 (1989)). Thus, the PAI-1 gene may serve as a genetic marker, or biomarker, for such diseases.

However, the traditional tools and methods of detecting the presence or absence of biomarkers, such as in situ hybridization and reverse transcriptase PCR (RT-PCR), are of limited utility because of the current inefficiencies in high-throughput screening of cardiac tissue samples, the lack of a panel of tissue samples representing different cardiovascular disease states, and the inability to simultaneously probe numerous tissue samples for the presence or absence of biomarkers associated with cardiovascular disease. The present invention provides tools and methods whereby a plurality of cardiovascular tissue can be rapidly screened for the presence, absence, or a variation in the expression of numerous biomarkers associated with the progression of cardiovascular disease.

I. The Progression of Cardiovascular Disease

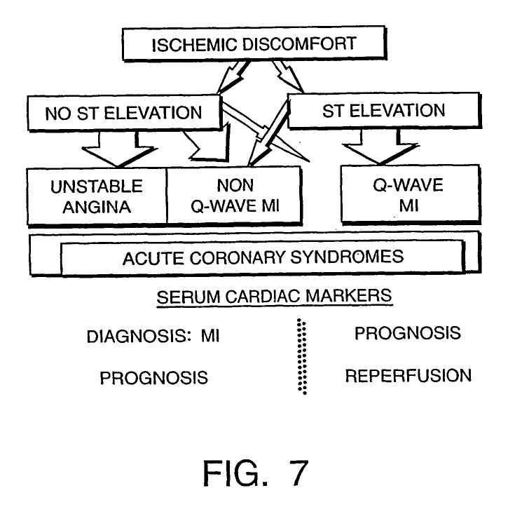

Although the exact mechanisms and steps involved in the progression of heart disease are not precisely known, it is believed that the progression follows the pathway of 1) atherosclerosis, 2) stable angina, 3) unstable angina, 4) non-Q-wave myocardial infarction, and 5) Q-wave myocardial infarction. (See FIG. 7). Patients with ischemic discomfort (i.e. discomfort due to inadequate circulation of blood to the myocardium) may present with or without ST-segment elevation on the electrocardiogram. The majority (large arrow) of patients with ST-segment elevation ultimately develop a Q-wave acute myocardial infarction (AMI), whereas a minority (small arrow) develop a non-Q-wave AMI. (See FIG. 7). Of patients who present without ST-segment elevation, the majority (large arrows) are ultimately diagnosed as having either unstable angina or non-Q-wave AMI based on the presence or absence of a cardiac marker such as the MB isozyme of creatine phosphokinase (CK-MB) detected in the serum (See, e.g., U.S. Pat. No. 5,137,609 to Manian et al., at column 8); a minority of such patients ultimately develop a Q-wave AMI. (See FIG. 7). The spectrum of clinical conditions ranging from unstable angina to non-Q-wave AMI and Q-wave AMI is referred to as acute coronary syndromes. (See Antman E M, Braunwald E., “Acute myocardial infarction,” in Heart Disease: A Textbook of Cardiovascular Medicine (1996), Philadelphia, Pa.: WB Saunders, Braunwald E B, editor). Various definitions of unstable angina have been proposed, but in 1989, Braunwald devised a classification system to ensure uniformity of categorization, as well as diagnostic and prognostic information. Braunwald E., “Unstable angina: a classification,” Circulation, 80: 410-414 (1989). This system is used to classify angina according to the severity of the chemical manifestation.

The clinical circumstances in which unstable angina develops, are defined as either angina in the presence or absence of other conditions (e.g., anemia, fever, hypoxia, tachycardia, or thyrotoxicosis), or angina within two weeks after an acute myocardial infarction; and whether or not electrocardiographic abnormalities are present. Given the heterogeneity of the clinical manifestations of unstable angina, it is not surprising that the prognosis is quite variable. Clinically speaking, patients with unstable angina and those with non-Q-Wave myocardial infarction often present in similar manner, and the distinction between the two conditions can be made only many hours or days later, when the results of cardiac-enzyme tests become available. (See Braunwald et al., “ACC/AHA guidelines for the management of patients with unstable angina and non-ST-segment elevation myocardial infarction: executive summary and recommendations: a report of the American College of Cardiology/American Heart Association Task Force on Practice Guidelines (Committee on the Management of Patients With Unstable Angina),” Circulation, 102: 1193-1209 (2000)). Thus, the development of diagnostic tools that would enable an earlier, pre-symptomatic detection of, and distinction between, underlying cardiovascular disease states is desirable. The compositions and methods of the present method provide a genetically-based tool that can be utilized to distinguish between the various stages in the progression of cardiovascular disease.

II. Construction of a Cardiovascular Tissue Microarray

Microarray technology has been developed in response to the need for simultaneous analysis of the thousands of genes. In a typical application, high-density nucleic acid samples, usually cDNAs or oligonucleotides, are delivered (or printed) by a robotic system onto very small, discrete areas of coated substrates, usually microscopic glass slides or membrane filters, and then immobilized to the substrate. The resulting microarray is then hybridized with a complex mixture of fluorescently labelled nucleic acids (probe) derived from a desired source. Following hybridization, the fluorescent markers are detected using a high resolution laser scanner. A gene pattern is obtained by analyzing the signal emitted from each spot with digital imaging software. In the case of gene expression analysis, the pattern of the experimental sample can be compared with that of a control for differential analysis.

Mutations and polymorphisms, in particular single nucleotide polymorphisms (SNPs), can be studied within and among species using high-density oligonucleotide arrays. These so-called mutation detection arrays consist of oligonucleotides representing all known sequence variants of a gene or a collection of genes. Because hybridization to oligonucleotides is sensitive enough to detect single-nucleotide mismatches, an homologous gene carrying an unknown sequence variation can be screened rapidly for a large number of changes.

The compositions of the present invention provide a tool comprising a cardiac tissue microarray that will allow the pre-symptomatic identification of cardiovascular disease, and the associated genetic risk factors. An example of tumor tissue microarray construction can be found in the Instruction Manual for the Beecher Instruments (Silver Spring, Md.) Tissue Arrayer.

In one embodiment, the present invention contemplates a composition for the detection of biomarkers associated with cardiovascular disease, comprising a tissue microarray, wherein said microarray comprises a plurality of human cardiovascular tissue samples placed on the surface of said microarray. Although it is not intended that the present invention be limited to any one particular method of preparing a cardiovascular tissue microarray, in one embodiment, the construction of said microarray involves the embedding of cardiovascular tissue in paraffin. In an alternative embodiment, the construction of said microarray involves the substitution of the paraffin embedding step above with the preparation of frozen cardiovascular tissue.

A. Preparation of Paraffin-Embedded Cardiovascular Tissue

Paraffin embedding is a process in which the tissue specimen is fixed to preserve its cellular structures, and blocked out and embedded in paraffin to stabilize it for long-term storage and easy sectioning or microdissection. Although it is not intended that the present invention be limited to a specific method by which cardiovascular tissue is embedded in paraffin, in one embodiment, the present invention contemplates the following method.

Cardiovascular tissue in a formaldehyde fixative solution (e.g. formalin) or RNAlater™ (Ambion Cat. No. 7020) is processed by sequential incubations in various concentrations of 1) ethanol, 2) xylenes, and then embedded in paraffin. The paraffin embedded tissues are then cut into sections, with a typical thickness of between 1-10 microns (e.g 5.5 microns) on a clean microtome with a clean blade to produce paraffin ribbons containing the tissues. The paraffin ribbons are floated in RNAse-free, deionized water at 43-44° C. The paraffin ribbons are then mounted on plain, uncoated glass microscope slides. Prior to subjecting the paraffin embedded tissues to screening for the presence or absence of biomarkers associated with cardiovascular disease, the paraffin is removed from the tissue sections by sequential incubations in various concentrations of 1) xylenes, 2) ethanol, and 3) distilled water.

B. Preparation of Frozen Cardiovascular Tissue

Cardiovascular frozen tissue storage is another way to preserve specimens and stabilize them for long-term storage and sectioning, and is a well-know method in the field. Tissue is embedded in a viscous compound, such as OCT compound (Tissue-Tek Cat. No. 4583) and deep-frozen on dry ice, or at a lower temperature (e.g. −125° C.). The block is removed from the cryomold and attached to a cryostat with OCT. The block is allowed to equilibrate to the cryostat temperature (−20° C.) for about 15 minutes. Tissue block sections are cut onto plain, uncoated glass slides.

C. Arrangement of Cardiovascular Tissues on a Microarray

It is not intended that the present invention be limited to any specific arrangement or configuration of cardiovascular tissue on a microarray. Moreover, it is not intended that the present invention be limited to any specific number or types of cardiovascular tissues arranged on a microarray.

In one embodiment, the arrangement of cardiovascular tissue comprises a plurality of samples of various cardiovascular tissue types from a single donor source, wherein each tissue represents a different state or stage of the progression of heart disease. In one embodiment, cardiovascular tissues are taken from a single donor's aorta, circumflex, and left coronary artery at separate times coinciding with the expression of symptoms related to atherosclerosis, stable angina, unstable angina, non-Q-wave myocardial infarction, and Q-wave myocardial infarction. Said tissues are conveniently arranged in paraffin or frozen embedding mold, as described above, and arrayed as described below.

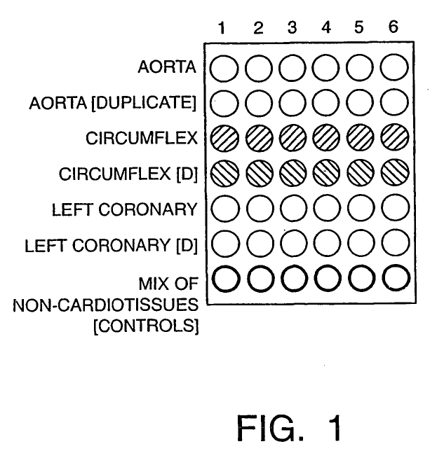

For example, FIG. 1 depicts a microarray comprised of cardiovascular tissue derived from a single donor with cardiovascular disease. The rows of said microarray depict four different tissues derived from the donor (e.g. aorta, circumflex, left-coronary artery, and a non-cardiovascular tissue control) arranged in duplicate. The columns of said microarray represent said tissues that were isolated from the donor as to correspond with different states or stages in the progression of cardiovascular disease (e.g. such as to form a single-source chronological microarray). Specifically, the tissues in Column 1 are non-diseased cardiovascular tissues. The tissues in Column 2 correspond to an atherosclerotic stage of disease. The tissues in Column 3 correspond to a stable angina stage of disease. The tissues in Column 4 correspond to an unstable angina stage of disease. The tissues in Column 5 correspond to non-Q-wave myocardial infarction stage of disease. The tissues in Column 6 correspond to Q-wave myocardial infarction stage of disease. The present invention also contemplates the arraying of additional cardiovascular tissues (e.g. arterial, venous, and cardiac) in the same manner as shown in FIG. 1 (whether as single samples, or in duplicate or triplicate).

It is not intended that the present invention be limited to a cardiovascular tissue microarray wherein said tissues are derived from a single donor. In one embodiment, cardiovascular tissue selected from the group comprising aorta, circumflex, and left coronary artery tissue, is derived from multiple donors at separate times coinciding with the expression of symptoms related to atherosclerosis, stable angina, unstable angina, non-Q-wave myocardial infarction, and Q-wave myocardial infarction. Said tissues are conveniently arrayed in paraffin or frozen embedding mold, as described above, and arrayed as described below.

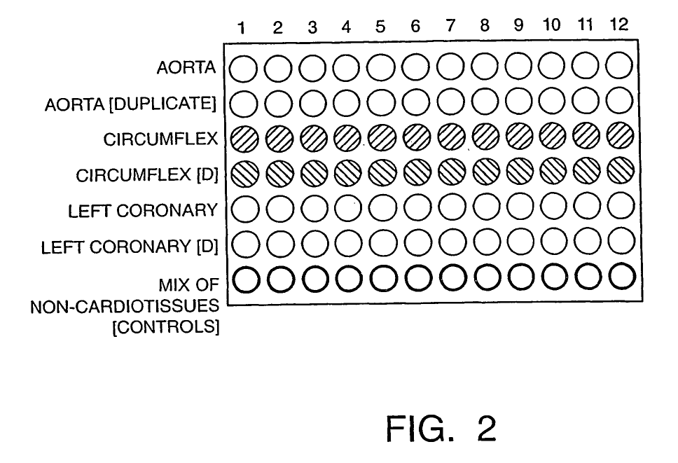

For example, FIG. 2 depicts a microarray comprised of cardiovascular tissue derived from a two donors having cardiovascular disease (e.g. such as to form a comparative chronological microarray). The rows of said microarray depict four different tissues derived from the two donors (e.g. aorta, circumflex, left-coronary artery, and a non-cardiovascular tissue control) arranged in duplicate. The columns of said microarray represent said tissues that were isolated from each donor as to correspond with different states or stages in the progression of cardiovascular disease. Columns 1-6 correspond to the tissues derived from a first donor, whereas Columns 7-12 correspond to tissues derived from a second donor. Specifically, the tissues in Columns 1 and-7 are non-diseased cardiovascular tissues. The tissues in Columns 2 and 8 correspond to a atherosclerotic stage of disease. The tissues in Columns 3 and 9 correspond to a stable angina stage of disease. The tissues in Columns 4 and 10 correspond to an unstable angina stage of disease. The tissues in Columns 5 and 11 correspond to non-Q-wave myocardial infarction stage of disease. The tissues in Columns 6 and 12 correspond to Q-wave myocardial infarction stage of disease. The present invention also contemplates the arraying of additional cardiovascular tissues (e.g. arterial, venous, and cardiac) in the same manner as shown in FIG. 2 (whether as single samples or in duplicate or triplicate).

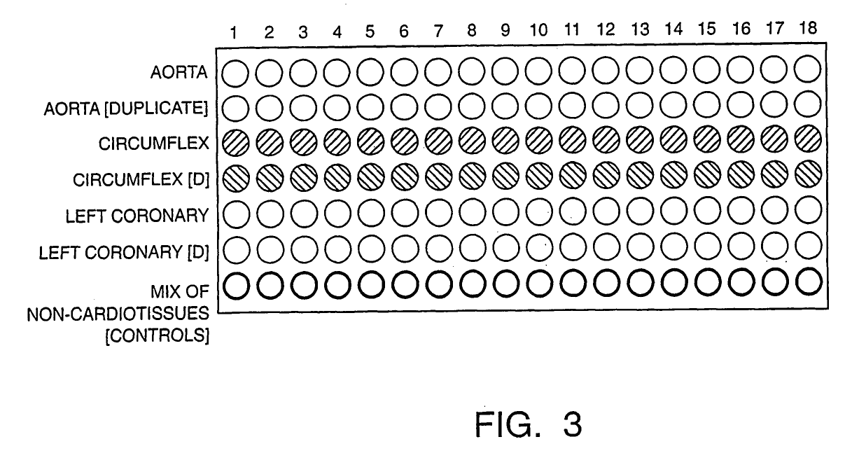

In another embodiment, a cardiovascular tissue microarray comprised of a plurality of tissues from more than two donors is contemplated. For example, FIG. 3 depicts a microarray comprised of cardiovascular tissue derived from a three donors having cardiovascular disease. The rows of said microarray depict four different tissues derived from the donor (e.g. aorta, circumflex, left-coronary artery, and a non-cardiovascular tissue control) arranged in duplicate. The columns of said microarray represent said tissues that were isolated from each donor as to correspond with different states or stages in the progression of cardiovascular disease. Columns 1-6 correspond to the tissues derived from a first donor.

Columns 7-12 correspond to tissues derived from a second donor, whereas Columns 13-18 correspond to said tissues from a third donor. Specifically, the tissues in Columns 1, 7, and 13 are non-diseased cardiovascular tissues. The tissues in Columns 2, 8, and 14 correspond to an atherosclerotic stage of disease. The tissues in Columns 3, 9 and 15 correspond to a stable angina stage of disease. The tissues in Columns 4, 10, and 16 correspond to an unstable angina stage of disease. The tissues in Columns 5, 11, and 17 correspond to non-Q-wave myocardial infarction stage of disease. The tissues in Columns 6, 12, and 18 correspond to Q-wave myocardial infarction stage of disease. The present invention also contemplates the arraying of additional cardiovascular tissues (e.g. arterial, venous, and cardiac) in the same manner as shown in FIG. 3 (whether as single samples, or in duplicate or triplicate).

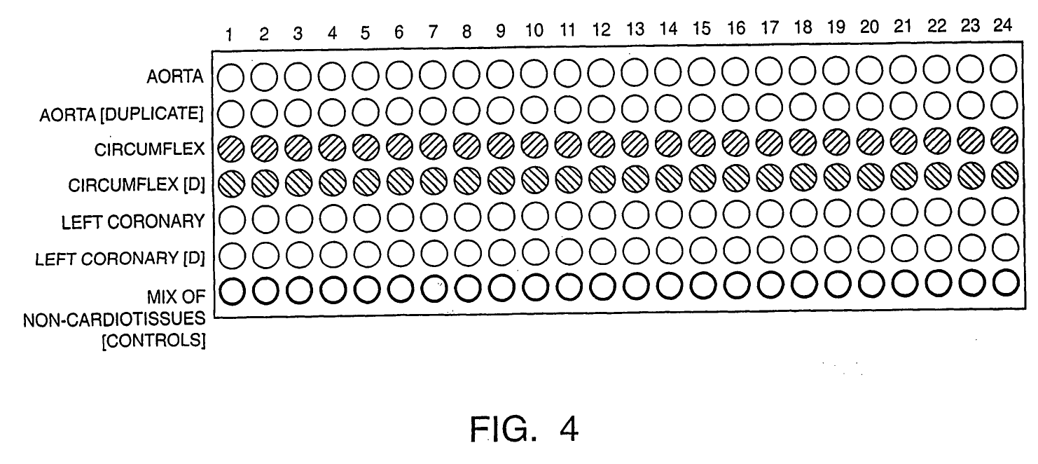

In yet another embodiment, a cardiovascular tissue microarray comprised of a plurality of tissues from more than three donors is contemplated. For example, FIG. 4 depicts a microarray comprised of cardiovascular tissue derived from a four donors having cardiovascular disease. The rows of said microarray depict four different tissues derived from the donor (e.g. aorta, circumflex, left-coronary artery, and a non-cardiovascular tissue control) arranged in duplicate. The columns of said microarray represent said tissues that were isolated from each donor as to correspond with different states or stages in the progression of cardiovascular disease. Columns 1-6 correspond to the tissues derived from a first donor having cardiovascular disease. Columns 7-12 correspond to tissues derived from a second donor having cardiovascular disease. Columns 13-18 correspond to tissues derived from a third donor having cardiovascular disease, whereas Columns 19-24 correspond to said tissues from a fourth donor having cardiovascular disease. Specifically, the tissues in Columns 1, 7, 13, and 19 are non-diseased cardiovascular tissues. The tissues in Columns 2, 8, 14, and 20 correspond to an atherosclerotic stage of disease. The tissues in Columns 3, 9, 15, and 21 correspond to a stable angina stage of disease. The tissues in Columns 4, 10, 16, and 22 correspond to an unstable angina stage of disease. The tissues in Columns 5, 11, 17, and 23 correspond to non-Q-wave myocardial infarction stage of disease. The tissues in Columns 6, 12, 18, and 24 correspond to Q-wave myocardial infarction stage of disease. The present invention also contemplates the arraying of additional cardiovascular tissues (e.g. arterial, venous, and cardiac) in the same manner as shown in FIG. 4 (whether as single samples, or in duplicate or triplicate).

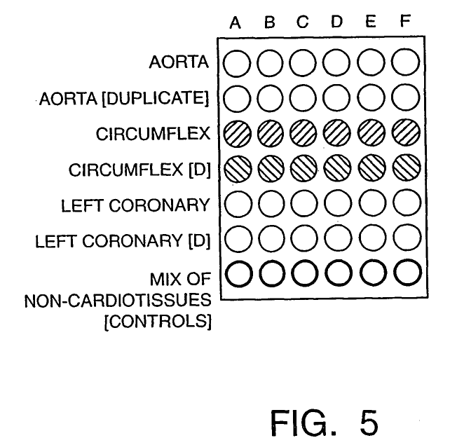

It is not intended that the present invention be limited to a microarray wherein said microarray is comprised of a plurality of cardiovascular tissues isolated from a donor as to correspond with multiple states or stages in the progression of cardiovascular disease. In one embodiment, the invention contemplates a comparative cardiovascular tissue microarray comprising tissues from multiple donors corresponding to a single stage (e.g. the atherosclerotic, stable angina, unstable angina, non-Q-wave myocardial infarction, or Q-wave myocardial infarction stage) of cardiovascular disease. For example, FIG. 5 depicts a comparative microarray comprised of cardiovascular tissue derived from a five different donors (corresponding to Columns B-F, respectively) suffering from cardiovascular disease. Column A of said microarray corresponds to non-diseased cardiovascular tissues. The rows of said microarray depict four different tissues derived from the donor (e.g. aorta, circumflex, left-coronary artery, and a non-cardiovascular tissue control) arranged in duplicate. In another embodiment, said comparative microarray comprises tissues from multiple donors corresponding to more than one disease state (e.g. as seen in FIGS. 1-4). The present invention also contemplates the arraying of additional cardiovascular tissues (e.g. arterial, venous, and cardiac) in the same manner as shown in FIG. 5 (whether as single samples, or in duplicate or triplicate).

It is not intended that the present invention be limited solely to the arrangement of diseased cardiovascular tissue on a microarray. In one embodiment, non-diseased Cardiovascular tissues, selected from the group comprising arterial, venous, aorta, circumflex, and left and right coronary artery tissue, is placed on the array, to act as a negative control. In a preferred embodiment, both diseased and non-diseased cardiovascular tissues selected from the group comprising aorta, circumflex, and left and right coronary artery tissue, are placed on the array.

It is not intended that the present invention be limited to having cardiovascular tissues arranged on a microarray in singlicate (i.e. a single sample or lane of tissue samples). In one embodiment, said cardiovascular tissues are arranged in duplicate. In another embodiment, said cardiovascular tissues are arranged in triplicate.

It is not intended that the present invention be limited to arranging cardiovascular tissue on a microarray in any specific direction. In one embodiment, said tissues representing different states of cardiovascular disease are arranged on the microarray sequentially from left to right (horizontally). In another embodiment, said tissues are arranged on the microarray sequentially from right to left (horizontally). In an alternative embodiment, said tissues representing different states of cardiovascular disease are arranged on the microarray sequentially from top to bottom (vertically). In a further alternative embodiment, said tissues are arranged on the microarray sequentially from bottom to top (vertically).

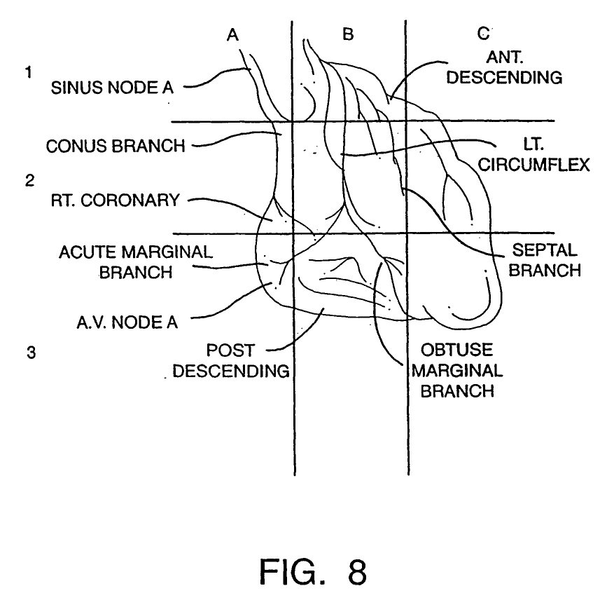

In preferred embodiments of the invention, functional and topological arterial tissue microarrays comprising a plurality of different cardiovascular tissues, and representing different cardiovascular disease states, are contemplated. For example, the present invention contemplates a cardiovascular tissue microarray based on the topological map of the coronary arterial tree as depicted in FIG. 8 (i.e. the array is organized so as to represent samples taken from particular portions of the heart).

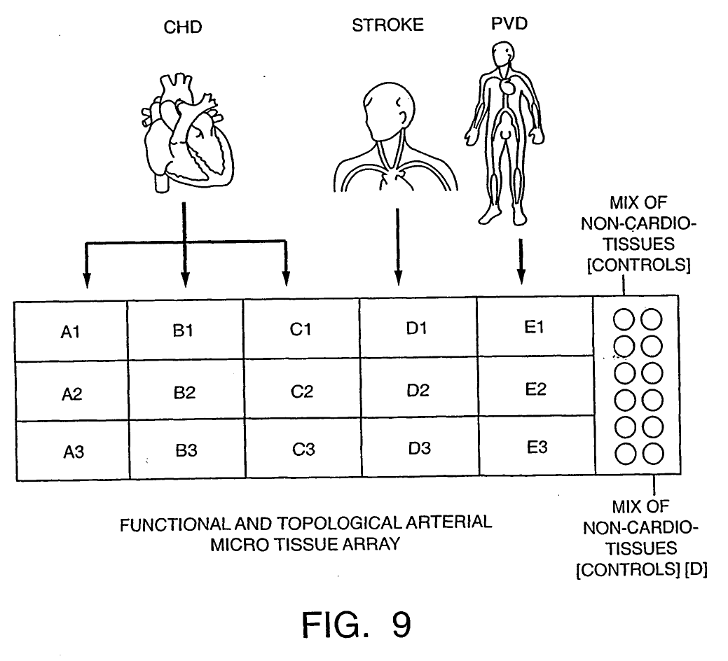

Specifically, FIG. 8 depicts a topological map of the coronary arterial tree as viewed in one of the projections commonly used in coronary arteriography. The map divides the human heart into discrete sections or regions (e.g. A-1, B-2, C-3, etc.) which correspond with identically numbered sections of functional and topological arterial tissue microarrays as described herein. (See FIGS. 9-14 for examples of tissue microarrays based on the topological map of FIG. 8).

The present invention also contemplates a functional and topological cardiovascular tissue microarray comprising diseased cardiovascular tissues, from different portions of the cardiovascular system (e.g. any of the tissues depicted in FIG. 8), and representing different clinical manifestations of atherosclerotic cardiovascular disease (e.g. coronary heart disease, stroke, and peripheral arterial vascular disease), as depicted in FIG. 9. Moreover, it is not intended that the present invention be limited to a functional and topological cardiovascular tissue microarray comprising tissue samples from a single donor. In another embodiment, said microarray comprises cardiovascular tissue samples from more than one donor.

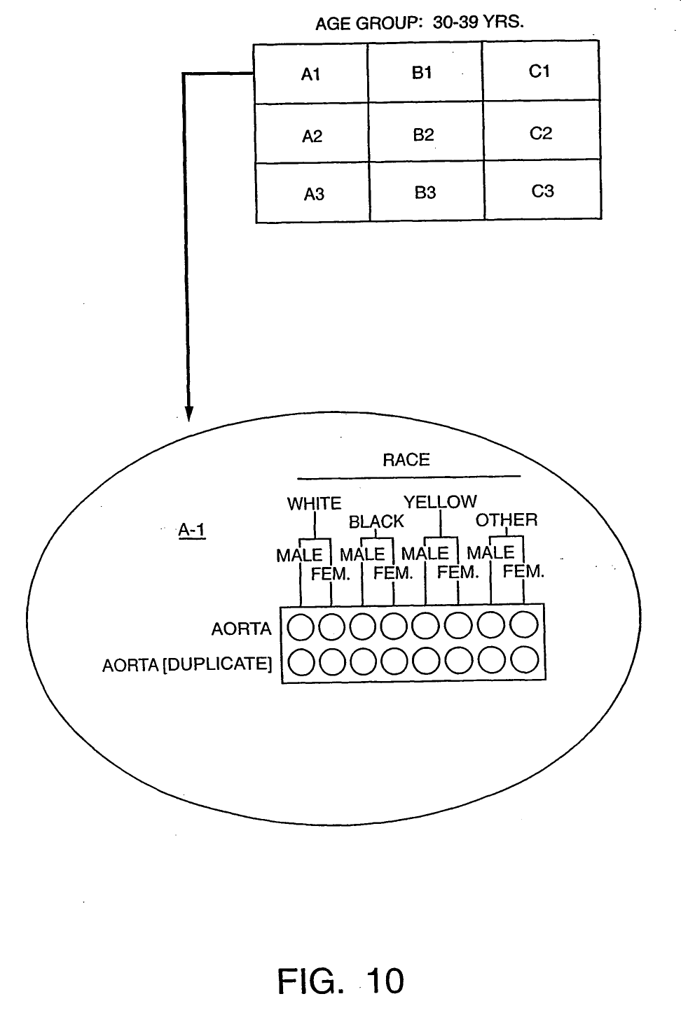

For example, as depicted in FIG. 10, the present invention contemplates one embodiment of a cardiovascular tissue microarray comprised of distinct cardiovascular tissue types. While only aorta is shown, such tissue types may be selected from the group consisting of: aorta, right atrium, and right ventricle (sections A-1 through A-3 respectively); circumflex, septal wall, and obtuse marginal branch of the left coronary artery (sections B-1 through B-3, respectively); and anterior descending branch of the left coronary artery, left atrium, and left ventricle (sections C-1 through C-3, respectively). The tissues depicted in sections A-C in FIG. 10 are those tissues associated with coronary heart disease. (See also FIG. 9). The cardiovascular tissue microarray of FIG. 10 may also comprise tissues selected from the group of sinuatrial nodal (sinus node) artery, conus arteriosus branch of the right coronary artery, right coronary artery, acute marginal branch of the right coronary artery, atrioventricular (A.V.) nodal artery, and posterior descending branch of the right coronary artery. Moreover, it is contemplated that a cardiovascular tissue microarray, as depicted in FIG. 10, be comprised of cardiovascular tissues obtained from donors (living or non-living) representing different age groups. However, it is not intended that said tissues be obtained from donors of any specific age group. For example, in one embodiment, cardiovascular tissues are obtained from donors between the ages of 30-39. In another embodiment, said donors are between the ages of 40-49. In another embodiment, said donors are between the ages of 50-59. In a further embodiment, said donors are between the ages of 60-69. Furthermore, the present invention is not limited to donors of any particular sex, race, or ethnic background. As shown in FIG. 10, donors of several races and ethnicities such as “white” (e.g. Caucasian, Arabian, and Middle Eastern/Southwest Asian), “black” (e.g African, African-American, West Indian, and Creole), “yellow” (e.g. Asian, Indian, and Pacific Islander) of both sexes, are contemplated. However, the present invention also contemplates donors of many “other” races and ethnicities such as, for example, Hispanic, Native Hawaiian, American Indian/Native American, Alaskan Native (e.g. Alaskan Indian, Aleut, Eskimo), Aboriginal/Indigenous Peoples, and multi-racial donors as well.

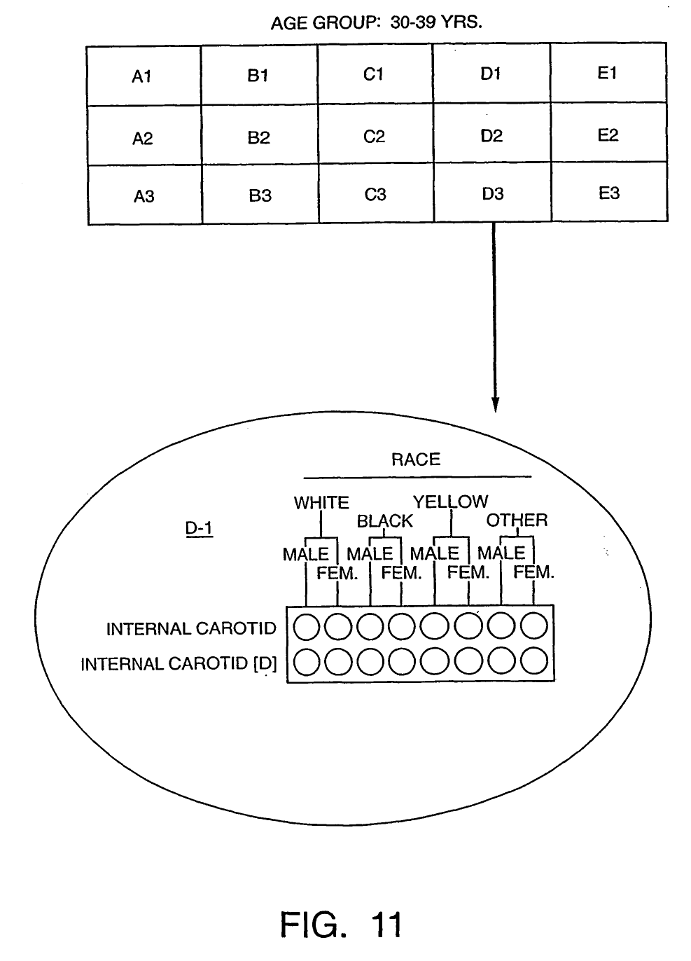

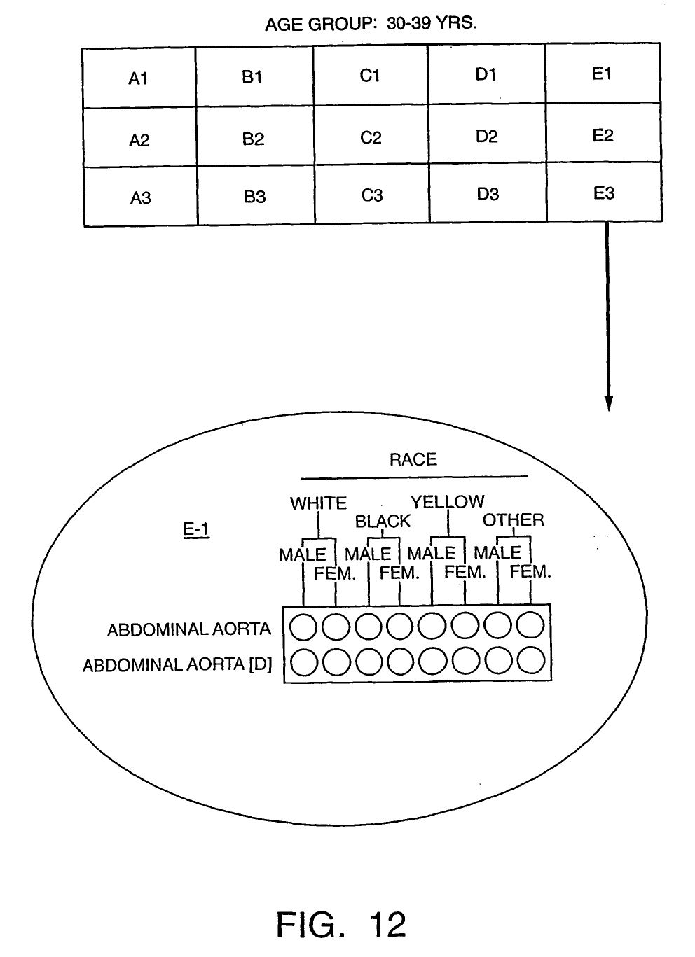

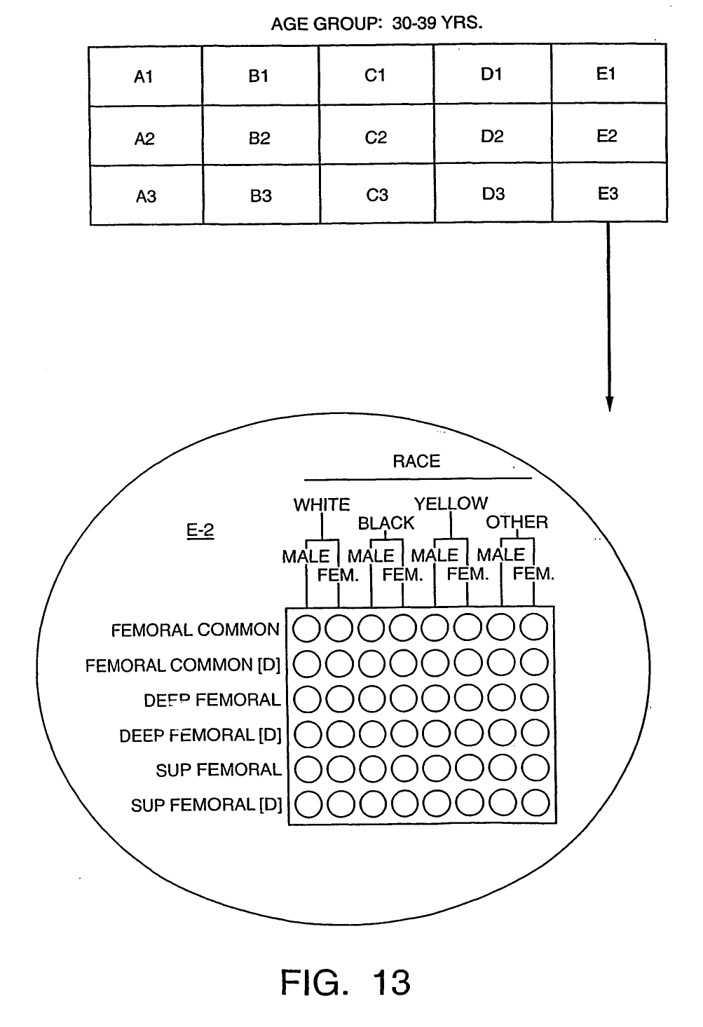

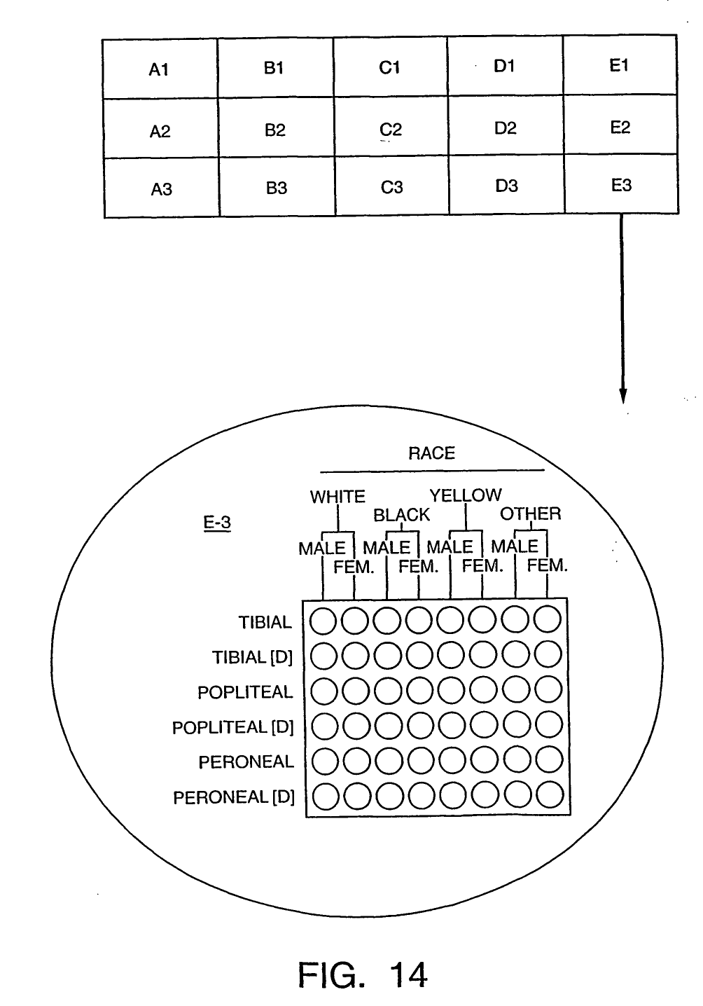

As depicted in FIG. 9, the present invention contemplates cardiovascular tissue microarrays in which more than one clinical manifestation of atherosclerotic cardiovascular disease is represented. For example, FIG. 11 depicts the cardiovascular tissue microarray of FIG. 10 further comprising cardiovascular tissues implicated in stroke as indicated by sections D-1 through D-3 (internal carotid, external carotid, and common carotid, respectively). In another embodiment, as depicted in FIG. 12, the present invention contemplates the cardiovascular tissue microarray of FIG. 10 further comprising cardiovascular tissues implicated in peripheral arterial vascular disease (PVD) as indicated by sections E-1 through E-3. As exemplified by FIG. 12, the present invention contemplates such a cardiovascular tissue microarray wherein section E-1 comprises abdominal aorta tissue specimens. As indicated by FIG. 13, present invention contemplates a cardiovascular tissue microarray wherein section E-2 comprises tissues selected from the group consisting of common femoral artery, deep femoral artery, and superior femoral artery. Moreover, as indicated by FIG. 14, present invention contemplates a cardiovascular tissue microarray wherein section E-3 comprises tissues selected from the group consisting of tibial artery, popliteal artery, and peroneal artery.

III. Detection of Biomarkers Associated With Cardiovascular Disease

A. General Overview

The present invention relates to an enhanced method of detecting biomarkers associated with cardiovascular disease through the use of cardiovascular tissue microarrays as described above. The methods of the present invention comprise the use of said cardiovascular tissue microarray such that a plurality of tissues can be rapidly screened for the presence or absence of numerous biomarkers associated with cardiovascular disease.

1. Screening of Cell-associated Markers of Risk of Cardiovascular Disease

Certain genetic or biomarkers associated with cardiovascular disease have been identified. As noted above, the expression of Serpine-1, the gene product of the plasnuogen activator inhibitor type 1 (PAI-1) gene, has been linked to cardiovascular diseases. Arterial serpins regulate central steps in the thrombotic and thrombolytic cascades. PAI-1 is produced by cells in the vessel wall and acts to inhibit plasminogen activators such as TPA. (See Lucas et al., “Transplant Vasculopathy: Viral Anti-inflammatory Serpin Regulation of: atherogenesis,” J Heart Lung Transplant, 19(11): 1029-38 (2000)). The specific inhibitors of plasminogen activators have been classified into 4 immunologically distinct groups: PAI-1 type PA inhibitor from endothelial cells, PAI-2 type PA inhibitor from placenta, monocytes, and macrophages; urinary inhibitor; and protease-nexin-I. PAI-1 cDNA encodes a protein containing 402 amino acids with a predicted non-glycosylated molecular mass of 45 kD. Cultured human umbilical vein endothelial cells contain 2 PAI-1 mRNA species, both encoded by a single gene, differing by 1 kb in the 3′ untranslated region. Plasminogen activator inhibitor shows structural similarities to angiotensinogen, alpha 1-antitrypsin and antithrombin III. The deduced amino acid sequence showed 30% homology with alpha-1-antitrypsin and antithrombin III, indicating that it is a member of the serine proteinase inhibitor (serpin) superfamily.

In Canneliet et al., “Inhibitory role of plasminogen activator inhibitor-1 in arterial wound healing and neointima formation: a gene targeting and gene transfer study in mice,” Circulation, 96(9): 3180-91 (1997), recombinant PAI-1 expression was demonstrated in injured arteries and was found to inhibit neointima formation by inhibiting smooth muscle cell migration, suggesting that this may have implications for the treatment of arterial stenosis in human following surgical intervention. Kohler and Grant, “Plasminogen-activator inhibitor type 1 and coronary artery disease,” N. Engl. J Med., 342(24): 1792-801 (2000), discussed the mechanisms regulating the production and action of PAI-1 and the role of gene-environment interactions in controlling fibrinolysis. They also discussed how these factors may affect the risk of artherothrombosis in persons with coronary artery disease. Thus, the PAI-1 gene may serve as a biomarker of cardiovascular disease, and may be detected as described below.

Endothelial Constitutive Nitric Oxide Synthase

Plasma NOx (nitrate and nitrite) is a stable end product of the vasodilator Nitric Oxide (NO). Several polymorphisms in the endothelial constitutive NO synthase (ecNOS) gene have been reported, including the 4a/4b VNTR polymorphism in intron 4, the E298D mutation in exon 7, and the G10-T polymorphism in intron 23. (See Yoon et al., “Plasma nitric oxide concentrations and nitric oxide synthase gene polymorphisms in coronary artery disease,” Clin. Chem., 46(10): 1626-30 (2000)). The aims of this study were to examine plasma NOx in patients with coronary artery disease (CAD) and to assess the association between plasma NOx concentrations and three ecNOS gene polymorphisms. Id.

Briefly, plasma NOx was measured in samples from 128 healthy controls and from 110 CAD patients at least two months after myocardial infarction. Id. Three genetic polymorphisms that are known, or have been suggested, to be associated with plasma NOx concentration were also analyzed by PCR-restriction fragment length polymorphism (PCR-RFLP). Id. The results of the analysis indicated that median plasma NOx was significantly higher (P<0.001) in CAD patients than in controls. Id. Furthermore, the median plasma NOx was significantly higher (P<0.001) in hypertensive CAD patients than in controls and normnotensive CAD patients. Id. The G-allele frequency of the G10-T polymorphism in intron 23 was was significantly higher in CAD patients than in controls. Id. Other polymorphisms showed no differences in allelic frequencies among the control and CAD groups. Id. In the controls, individuals with the E298D mutation in exon 7 showed significantly higher (P=0.001) median plasma NOx than those without the mutation. Id. Thus it was concluded that plasma NOx is higher in hypertensive CAD patients than in normnotensive CAD patients and controls, and that the E298D polymorphism of the ecNOS gene is associated with said increase in plasma NOx. Id.

It has also been reported that a mutation (−786T→C) in the promoter region of the endothelial nitric oxide synthase gene (eNOS) reduces transcription of the gene, and is strongly associated with coronary spastic angina and myocadial infarction. (See Miyamoto et al., “Replication protein A1 reduces transcription of the endothelial nitric oxide synthase gene containing a −786T→C mutation associated with coronary spastic angina,” Hum. Mol. Genet., 9(18): 2629-37 (2000)). The functional importance of the diminished eNOS expression was revealed by finding that serum nitrate/nitrite levels among individuals carrying the −786T→C mutation significantly lower than among those individuals without the mutation. Id.

2. Screening of Serum-associated Markers and Screening of Detection Polymorhism by Specific Gene(s) of Risk of Cardiovascular Disease

Screening of Serum-associated Markers of Risk of Cardiovascular Disease

Recent studies indicate that there are several serum-associated markers that can be directly correlated to both the risk of, as well as, the presence of, cardiovascular disease. It is believed that specific serum-associated markers can also be correlated to one or more specific stages of cardiovascular disease. For example, an increase in the expression level of the lipoprotein-associated phospholipase A2 (platelet-activating factor acetylhydrolase) shows a strong, positive association with the risk of coronary heart disease. Packard et al., “Lipoprotein-associated phospholipase A2 as an independent predictor of coronary heart disease,” New Engl J Med, 343 (16): 1148-1155 (2000). Similarly, it has been found that high levels of the plasma-associated proteins Troponin T (a marker of myocardial damage) and C-reactive protein (a marker of inflammation) are strongly related to the long-term risk of death from cardiac causes in patients suffering from unstable coronary artery disease. Lindahl et al., “Markers of Myocardial Damage and Inflammation in Relation to Long-Term Mortality in Unstable Coronary Artery Disease,” New Engl J Med, 343 (16): 1139-1147 (2000). For example, a subject who presents with a high level of Troponin T, C-reactive protein, or lipoprotein-associated phospholipase A2 as positive indicators of the presence of cardiovascular disease can be selected for tissue biopsy and screening for similar cell and/or tissue-associated biomarkers. Finally, it has been shown that low density lipoprotein (LDL) and oxidized LDL downregulate the level of the key enzyme in endothelin-1 (ET-1) generation, endothelin-converting enzyme (ECE), in human internal mammary artery endothelial cells. Ruschitzka et al., “Tissue endothelin-converting enzyme activity correlates with cardiovascular risk factors in coronary artery disease,” Circulation, 102(10): 1086-92 (2000). The vascular ECE activity is inversely correlated with serum LDL levels and blood pressure, and positively associated with fibrinogen in human vascular tissue. Id. Hence, ECE activity may modulate cardiovascular risk in patients with coronary artery disease.

The present invention contemplates utilizing such serum-associated biomarkers in order to determine whether a test subject has, or does not have, cardiovascular disease. Specifically, the present invention contemplates that a test subject having a serum Troponin T level of greater than or equal to 0.60 μg/liter is determined to have an increased risk of death from cardiac causes. (See Lindahl et al., supra). Similarly, the present invention also contemplates that a test subject having a serum C-reactive protein level of greater than or equal to 10 mg/liter is determined to have an increased risk of death from cardiac causes. Id. Screening of detection Polymorhism by specific gene(s) of risk of cardiovascular disease The co-existence of multiple alleles at a locus is called genetic polymorphism. Any site at which multiple alleles exist as stable components of the population is by definition polymorphic. An allele is usually defined as polymorphic when it is present at a frequency of >1% in the population. Multiple versions of the wild-type allele may be distinguished by differences in sequence that do not affect their function, and which, therefore, do not produce phenotypic variants. As noted by the examples below, many different sequence variants may exist at a given locus, including those that change DNA sequence but do not change protein sequence, those that change protein sequence without changing function, those that create mutant proteins that are nonfunctional.

Apolipoprotein E

For example, Apolipoprotein E (APOE) is a major protein in lipid metabolism existing in three common isoform: APOE2, -3 and -4. The varepsilon4 alelle of the APOE gene coding for the APOE4 isoform is associated with an increased risk of myocardial infarction (MI) and of Alzheimer's disease. The promoter polymorphism −219 T alelle was associated with a significant increased risk of MI and the effect was shown to be independent of the presence of the other mutations, including the APOE epsilon2/epsilon3/epsilon 4 polymorphism. Moreover, the −219T allele greatly decreased the APOE plasma concentrations in a dose-dependent manner Scuteri et al., “Is the apoE4 allele an independent predictor of coronary events?,” Am J Med 110 (1): 28-32 (2001); Brazier et al., “Sequence polymorphisms in the apolipoprotein(a) gene and their association with lipoprotein(a) levels and myocardial infarction. The ECTIM Study,” Atherosclerosis 144(2):323-33 (1999).

Angiotensin I Converting Enzyme

Another example is the Angiotensin I converting enzyme (ACE). The enzyme is a dipeptidyl carboxypeptidase that plays an important role in blood pressure regulation and electrolyte balance by hydrolyzing angiotensin I into angiotensin II, a potent vasopressor, and aldosterone-stimulating peptide. The enzyme is also able to inactivate bradykinin, a potent vasodilator. The ACE gene encodes 2 isozymes. The somatic ACE isozyme is expressed in many tissues, including vascular endothelial cells, renal epithelial cells, and testicular Leydig cells, whereas the testicular or germinal ACE isozyme is expressed only in sperm. The importance of ACE in circulatory homeostasis is well documented. Besides being present as a membrane-bound enzyme on the surface of vascular endothelial cells, ACE also circulates in Miasma. The plasma enzyme may be synthesized in vascular endothelium. The inter-individual variability of plasma ACE concentration is determined by an insertion (I)/deletion (D) polymorphism situation in intron 16 of the ACE gene and known as the ACE/ID polymorphism.

In one study comparing patients after myocardial infarction (MI) with controls, an association was found between coronary heart disease and a polymorphism, ACE/ID, in the ACE gene. The insertion(l)/deletion (D) polymorphism involving about 250 bp situated in intron 16 of the ACE gene, the so-called ACE/ID polymorphism. Cambien et al., “Deletion polymorphism in the gene for angiotensin-converting enzyme is a potent risk factor for myocardial infarction,” Nature, 359 (6396): 641-4 (1992).

Factor VII Gene Polymorphism

Thrombosis underlies most acute manifestations of coronary atherosclerotic disease, including, but not limited to, myocardial infarction. Plaque disruption, with resulting exposure of tissue factor to blood and binding of tissue factor to circulating coagulation Factor VII, is considered a major cause of thrombosis in myocardial infarction. Several papers show that a high plasma level of Factor VII was a predictor of death due to coronary disease. Population studies have suggested that two common polymorphisms in the Factor VII gene -the substitution of glutamine for arginine at position 353 in the catalytic domain (R353Q) and a 10-bp insertion in the promoter region (5′F7)—maybe responsible for up to one third of the variation in Factor VII levels. The Factor VII gene is also characterized by a polymorphism involving a variable number of 37-bp repeats in intron 7 (IVS7). The rare alleles of each polymorphism are generally associated with decreased levels of Factor VII. It is biologically plausible that Factor VII does not influence the development of coronary atherosclerosis, but only its thrombotic complication, myocardial infarction. Russo et al., “Polymorphisms in the Factor VII gene and the risk of myocardial infarction in patients with coronary artery disease,” N. Engl. J Med. 343 (11): 774-80 (2000).

Plasma Homocysteine

Elevated plasma homocysteine level is an independent risk factor for cardiovascular disease. A common mutation, nucleotide 677C-T, in the gene coding for methylene tetrahydrofolate reductase (MTHFR) has been reported to reduce the enzymatic activity of MTHFR and is associated with elevated plasma levels of homocysteine, especially in subjects with low folate intake. (See Gulec et al., “Methylenetetrahydrofolate reductase gene polymorphism and risk of premature myocardial infarction,” Clin. Cardiol., 24(4): 281-84 (2001)).

E-Selectin Polymorphism

The functional consequences of the single amino acid substitution in E-selectin that resulting from a common S128R polymorphism in the human population has been shown. Wenzel et al., “E-selectin polymorphism and atherosclerosis: an associated study,” Hum. Mol. Genet., 3: 1935-37 (1994). For example, neutrophils rolling over CHO cells (expressing wild-type E-selectin) under shear stress showed twice as high a rate of arrest on S128R E-selectin. Id. This difference in leukocyte adhesion in vitro may have relevance in human atherosclerosis because the S128R E-selectin polymorphism has been associated with an increased incidence of early severe coronary artery disease. Id.

The present invention contemplates utilizing such gene polymorphisms in order to determine whether a test subject has, or does not have, cardiovascular disease.

B. Analysis

It is not intended that the present invention be limited to any specific method for the detection of biomarkers associated with cardiovascular disease. In one embodiment, such a method comprising: a) providing the cardiovascular tissue microarray of the present invention; and b) subjecting said microarray to analysis by a method selected from the group of histological analysis, immunological analysis, and nucleic acid hybridization analysis, such that the presence or absence of a biomarker associated with cardiovascular disease is determined, is contemplated.

1. Histological Analysis

In one embodiment, such a method comprising: a) providing the cardiovascular tissue microarray of the present invention; and b) subjecting said microarray histological analysis such that the presence or absence of a biomarker associated with cardiovascular disease is determined, is contemplated. For example, in one embodiment, said histological analysis comprises histological staining with hematoxylin and eosin as follows.

The cardiovascular tissue samples are sectioned and mounted in paraffin as described above in Part II.A. The paraffin is removed from the cardiovascular tissue paraffin sections prior to staining as described above in Part II.A. The de-paraffinzed tissue microarray is stained at room temperature by performing sequential incubations in ethanol, deionized water (dH₂0), Hematoxylin, Bluing Reagent, Eosin, and fixed with ethanol and xylene. The stained tissue microarray is air-dried and stored in a desiccator until used.

In another embodiment, said histological analysis is selected from the group of light microscopy, phase-contrast microscopy, and osmium tetroxide/glutaraldehyde treatment followed by electron microscopy, the methods of which are all well-characterized and known to those practiced in the field of art.

2. Immunological Analysis

In one embodiment, a method comprising: a) providing the cardiovascular tissue microarray of the present invention; and b) subjecting said microarray to immunological analysis such that the presence or absence of a biomarker associated with cardiovascular disease is determined, is contemplated. For example, in one embodiment, a cardiovascular tissue microarray is analyzed for the presence or absence of the Serpine-1 (Serp-1) protein (gene product of the PAI-1 gene) following the histological and immunocytochemical methods of van Gorder et al., “Cynomolgus Polyoma Virus Infection: A New Member of the Polyoma Virus Family Causes Interstitial Nephritis, Ureteritis, and Enteritis in Immunosuppressed Cynomolgus Monkeys,” Am J Pathology, 54(4): 1273-84 (1999).

Specifically, a plurality of cardiovascular tissues are fixed and embedded in paraffin as described above. Sections of the paraffin embedded tissue are sequentially stained with hematoxylin, eosin, and periodic acid Schiff-base (PAS). Said sections are mounted on plain, uncoated microscope slides, de-paraffinized in xylene, rehydrated in ethanol and phosphate buffered saline (PBS), and incubated in peroxide and methanol to block the activity of endogenous peroxidase. Said tissue sections are heat-treated, followed by incubation with avidin D and biotin to block endogenous biotin. The tissue-mounted slides are stained overnight with a primary monoclonal antibody to the Serpine-1 protein (PAI-1 antibody, Cat.No. AB 6383-ca-1020b, CamBio, Ltd., Cambridge, UK), followed by biotinylated horse anti-mouse IgG secondary antibody(Vector Laboratories, Burlingame, Calif.), then incubated in preformed avidin-biotinylated horseradish peroxidase complexes (Elite ABC, Vector Laboratories). Said tissue sections are washed and developed with 3-amino-9-ethyl-carbazole (Aldrich Chemicals, Milwaukee, Wis.), counterstained with Gill's hematoxylin, and mounted in glycergel (DAKO, Carpinteria, Calif.). The binding of the Serpine-1-specific monoclonal antibody to tissue samples arranged on a cardiovascular tissue microarray is visualized by microscopy, wherein the binding of said antibody indicates the expression of the PAI-1 gene product, and thus, the presence of a genetic marker associated with cardiovascular diseases.

3. Nucleic Acid Analysis

In one embodiment, a method comprising: a) providing the cardiovascular tissue microarray of the present invention; and b) subjecting said microarray to nucleic acid hybridization analysis, such that the presence or absence of a biomarker associated with cardiovascular disease is determined, is contemplated. In one another embodiment, said nucleic acid hybridization analysis comprises a nucleic acid hybridization method selected from the group of fluorescence in situ hybridization (FISH) and inz situ RT-PCR as described below.

a. Fluorescence In Situ Hybridization (FISH)

Of the various DNA mapping techniques currently available, fluorescence in situ hybridization (FISH) has proven to be very versatile because of its direct nature and sensitivity, its ability to visualize multiple targets in different colors simultaneously and its potential to cover a wide range of genomic resolutions. See Florijn el al., Human Mol. Genet. 4: 831-36 (1995).

FISH is an analytical technique used to visualize labeled DNA probes in the fluorescence microscope after binding to essentially complementary DNA molecules. The relative location of the bound probes is measured by digital image analysis techniques on images recorded from the fluorescence microscope.

It is not intended that the present invention be limited to any specific method utilizing the FISH technique to determine the presence or absence of biomarkers associated with cardiovascular disease in tissues on a cardiovascular microarray. In one embodiment, the present invention contemplates such a method for the detection of the presence or absence of the PAI-1 gene on a cardiovascular tissue microarray as follows.

Probe Generation and Nucleic Acid Hybridization

Probes are prepared by labeling DNA containing the nucleic acid sequence encoding the human PAI-1 gene using random priming, or in vitro DNA amplification using the polymerase chain reaction (PCR). Plasmid and PI DNA for probe preparation was isolated by standard alkaline lysis procedures and YAC DNA was prepared from yeast clones using standard protocols. (See Sherman, et al., Laboratory Course Manual for Methods in Yeast Genetics, Cold Spring Harbor Laboratory Press, NY (1986)). Probes can be labeled using a variety of haptens including haptens for non-isotopical labeling such as biotin, digoxigenin and fluorescein isothiocyanate (FITC). Probes are labeled with the haptens by incorporation of commercially available deoxynucleotide derivatives to which the haptens are bound covalently (e.g., fluorescein-dUTP, biotin-14-dCTP, digoxigenin-dUTP). The FITC-labeled probes can be seen in the fluorescence microscope by eye when bound in sufficient quantities or after immunocytochemical signal amplification using antibodies against FITC. The two indirect DNA labeling systems (biotin and digoxigenin) require post-hybridization detection of bound probe with affinity reagents (e.g., avidin, antibodies) carrying fluorochromes.

It is not intended that the present invention be limited a FISH method wherein a DNA probe is only labeled once. The present invention also contemplates both dual and triple probe-labeling schemes. For example, the dual-label probe labeling scheme contemplates the preparation of a biotinylated probe that bind to the PAI-1 gene, while another probe to a different biomarker of cardiovascular disease is labeled with digoxigenin and FITC, respectively. The bound probes are detected after hybridization by incubation with AMCA-avidin (blue fluorescence) for biotin-labeled probes, rhodamine-labeled sheep antibodies against digoxigenin (rhodamine-anti-digoxigenin; red fluorescence) for digoxigenin-labeled probes, or a mouse antibody against FITC followed by incubation with an FITC-conjugated horse-anti-mouse antibody (green fluorescence) for fluorescein-labeled probes. The red (digoxigenin) signal is typically amplified by incubation of the slide (washed in three changes of 2× Sodium Salt Citrate) with a rhodamine-labeled rabbit-anti-sheep antibody. Visualization of the AMCA signal involves two signal amplification steps using a biotinylated goat-anti-avidin antibody followed by incubation with AMCA-avidin.

The dual label/dual color scheme involves the labeling of probe DNAs with biotin or digoxigenin and bound probes are detected with avidin-FITC and rhodarmine-anti-digoxigenin. Hybridization signals are then amplified once with biotinylated goat-anti-avidin, followed by a second layer of avidin-FITC and a Texas Red-labeled antibody against sheep IgG.

FISH analysis requires the DNA probe as well as the target to be single stranded for hybridization. An efficient protocol for denaturation/hybridization is the application of approximately 20 ng/μl of each probe in a solution containing 55% formamide, 10% dextran sulfate, 100 ng/μl salmnon sperm DNA, 2× Sodium Salt Citrate to the slide, followed by the placement of a non-silanated coverslip on top. The cardiovascular tissues arranged on the microarray and probe(s) are simultaneously denatured by incubation at 95-100° C. on a hot plate. The hybridizations are allowed to proceed overnight at 37° C. and the slides are then washed in 2× Sodium Salt Citrate at 20° C. Bound PAI-1-specific probes are detected by conjugation with fluorochrome-labeled avidin and antibodies as describe above. Final washes of slides are done in 2× Sodium Salt Citrate, before they are mounted in anti-fade mounting medium (Vectashield; Vector Labs, Burlingame, Calif.) for microscopic inspection and subjected to image analysis as described below.

Image Analysis

A computer-assisted fluorescence microscope is used for multi-color visualization of DNA molecules after FISH. The system consists of a Leica DM IRB research microscope equipped with a CCD camera, and Kappa software.

The essential optical feature of the microscope is the use of a multi-band beam splitter and emission filter, and a computer-controlled filter wheel to change the excitation filters. Each fluorochrome in the specimen is excited by selecting the appropriate excitation filter. The band passes in the beam splitter and emission filter are such that all of the fluorochrome-specific images can be obtained without moving any elements in the imaging pathway. The registration shifts between the red and green images are less that 0.1 μm (referred to the object) at all points in the digital image (Mascio, et al., Cytometry 19: 51 (1995)). The current filters are capable of excitation in single bands centered around 360 nm, 405 nm, 490 nm, and 560 nm, and visualization simultaneously in multiple bands in the vicinities of 450 nm (blue), 520 nm (green), and 600 nm (red). In addition, dual band excitation filters for simultaneous observation of FITC/Texas red are employed (Sakamoto, 1995 #981). Tissue samples on a cardiovascular tissue microarray molecules show blue, red, and green fluorescing domains wherein they contain regions homologous to the probes used. For example, cardiovascular tissues in a microarray that contain regions homologous to the PAI-1 gene hybridize to, and therefore are detected by, a complementary biotinylated PAI-1 DNA probe, and are indicated by a blue, fluorescent signal. Cardiovascular tissue samples on a microarray containing an increase or decrease in the copy number of the PAI-1 gene over the baseline number are distinguishable from tissue samples including one copy, or zero copies, of the gene.

b. In Situ RT-PCR

In situ RT-PCR is an enabling technology for both amplifying and localizing target nucleic acid sequences to individual intact cells. The technique involves the amplification of mRNA sequences in cells and tissues specimens by firstly creating a complementary DNA (cDNA) template using reverse transcriptase (RT) and then amplifying the newly created DNA template, using either a labelled (e.g. with Digoxigenin or biotin) primer or labelled oligonucleotide (dUTP) within the PCR reaction mix. The labelled product is then detected using standard detection techniques as for conventional in situ hybridization or immunocytochemistry.

Although it is not intended that the present invention be limited to any specific method of nucleic acid hybridization analysis using in situ RT-PCR, in one embodiment, a method for the in situ RT-PCR of human total RNA to detect the presence, absence, or variation in the expression level of the human PAI-1 gene is conducted as follows. (See also, H. Iwata & J. Stegeman, “In situ RT-PCR detection of CYP1A mRNA in pharyngeal epithelium and chondroid cells from chemically untreated fish: involvement in vertebrate craniofacial skeletal development?,” Biochem Biophys Res Commun, 271(1): 130-37 (2000); K. Iijima et al., “Activation-induced expression of vascular permeability factor by human peripheral T cells: a non-radioisotopic semi-quantitative reverse transcription-polymerase chain reaction assay,” J Immunol Methods, 196: 199-209 (1996)). The PAI-1 primer set utilized is designed to detect mRNA encoding the Serpine-1 protein.

Sections of diseases and non-diseased human cardiovascular tissue are mounted on uncoated slides and dried at room temperature as described below in Example 1. In one embodiment, the following six slides (APES pre-coated) were prepared using aterial tissue (e.g. coronary artery and aorta) and cardiac tissue (e.g. ventricle and auricule):

Slides:

1) USH#I Cardiac tissue (Ventricle and Auricule) stored in RNAlater™+formalin-fixed 2hs+Paraffin-embedded

2) USH#2 Cardiac tissue (Ventricle and Auricule) stored and fixed in Formol+Paraffin-embedded

3) USH#3 Cardiac tissue (Ventricle and Auricule) fresh frozen tissue+cryostat sections

4) USH#4 Artery tissue (Coronary and Aorta) stored in RNAlater™+formalin-fixed 2hs+Paraffin-embedded

5) USH#5 Artery tissue (Coronary and Aorta) stored and fixed in Formol+Paraffin-embedded

6) USH#6 Artery tissue (Coronary and Aorta) fresh frozen tissue+cryostat sections

The slides were deparaffinized by immersion in xylenes at 37° C. for thirty minutes, immersion in xylenes at room temperature for ten minutes, followed by dehydrationin 100% ethanol at room temperature for ten minutes. The slides were transfered into fresh 100% Ethanol prior to rehydration. Rehydration at room temperature was accomplished by incubating the slides as follows: 1) 100% Ethanol for 2 minutes; 2) 95% Ethanol for 2 minutes; 3) 70% Ethanol for 2 minutes; and 4) Distilled Water for 4 minutes. The slides were air-dried on a paper towel (sample side up), immersed in 0.02 M HCL for ten minutes, and washed twice with Phosphate Buffered saline (PBS). The slides were extracted with 0.01% Triton X-100 in PBS for thirty minutes and washed twice for five minutes in PBS.

The slides were mildly digested for twenty minutes at 37° C. with Proteinase K (0.1 mg/ml in 50 mM Tris-HCI pH 7.6, 5 mM EDTA; Sigma Chemical, St. Louis, Mo.). The slides were washed twice for five minutes with PBS containing 2 mg/ml glycine. The slides were immersed in aqueous 20% acetic acid for 15 seconds at 4° C. in order to block endogenous alkaline phosphatase activity. The slides were washed twice for ten minutes with PBS and dehydrated by immersion in a graded ethanol series comprising 50% ethanol, 95% ethanol, and 100% ethanol respectively.

Parallel solution-phase PCR was performed for each “in situ RT-PCR assay.” All reaction set-ups require reference control genes for the target sequence. The following genes were amplified as reference controls: 1) Human Protein Phosphatase 1 (Low number copies); 2) Human Cytoskeletal Gamma Actin (Medium number copies); 3) Human cytoskeletal Beta-Actin (High number copies); and 4) Glycerlaldehyde 3-Phosphate Dehydrogenase (GAPDH). The procedure was based on the GeneAmp in Situ PCR Core Kit protocol using the GeneAmp In Situ PCR System 1000 (AB Cat. Nos. N808-0197 & N804-0001 respectively). The results of the solution-phase RT-PCR performed on RNA isolated and purified from cardiovascular tissue yielded the following PCR products: 1) the GAPDH gene (556 bp); 2) the PAI-1 gene (314 bp); 3) the Beta-actin gene (445 bp); 4) the Gamma-actin gene (275 bp); and 5) the Phosphatase 1 gene (394 bp).

A reverse transcriptase solution containing 10 μl of 5× Superscript First Strand buffer (Life Technologies, Inc.), 5 μl of 100 mM DTT, 2.5 μl of 10 mM dNTP mixture, 25 pM Oligo d(T)₁₆, 80 units RNAsin (Promega Cat. No. N2111), 50 units Superscript RNase H-Reverse Transcriptase (Life Technologies Cat. No. 18053017), and sterile water was applied to each slide. The sealed slides are placed on a heat block of the PCR Thermal Cycler (of the GeneAmp In Situ PCR System 1000) and incubated according to a temperature program as follows: 42° C. for 5 minutes; 50° C. for 50 minutes; 70° C. for 15 minutes; and held at 4° C. until removed. After incubation, the solution in the incubation chambers is removed, and the section is washed in Phosphate-Buffered Saline (PBS).

After reverse transcription, a PCR mixture comprising 0.5 μM of specific primers (See Example 11), 1×PCR buffer II (without MgCl₂), 3.0 mM MgCl₂, 0.2 mM dNTP mixture (0.2 mM each of DATP, dCTP, dGTP, plus 0.13 mM dTTP and 0.07 mM digoxigenin (DIG)-11-dUTP [Roche Molecular Cat. No. 1209256)), and 10 units AmpliTaq IS (AB Cat. No. N80S-0197) is applied to each section in Amplicover Clip/Amplicover Disc assembly chambers (AB Cat. Nos. N804-0501 & N8040600, respectively) on the slides. The sealed slides were placed on a heat block of the PCR Thermal Cycler and incubated according to the following thermal cycling program (denaturation, annealing, and extension respectively): 1 cycle at 92° C. for 1.5 minutes, followed by 35 cycles comprising 92° C. for 45 seconds, 46° C. for 1.5 minutes, and 72° C. for 1.5 minutes.

After labeling the PCR products, the slides are washed in Tris-NaCl buffer, and immersed in Blocking Reagent (Roche Molecular Cat. No. 1096176). The detection of the incorporated DIG-labeled dUTP (and thus, the presence, absence, or variation in the expression of, the PAI-1 gene) is performed with a highly specific anti-DIG antibody (Roche Molecular Cat. No. 1333062) conjugated with alkaline phosphatase solution (Roche Molecular Cat. No. 1464752), which is diluted in blocking reagent. The slides are incubated with the antibody/blocking reagent mixture, followed by visualization of the PAI-1-specific RT-PCR product by incubation of the slides in nitroblue tetrazolium/5-bromo-4-chloro-3-indolylphosphate (Promega Cat. No. S3771).

The results of the in situ RT-PCR of the PAI-1 gene from diseased (i.e. atherosclerotic) aortic tissue gave a strong positive signal after staining (as described herein). However, no signal was observed in the results of the in situ RT-PCR of the PAI-1 gene from non-diseased (i.e. non-atherosclerotic) aortic tissue after staining. Moreover, in situ RT-PCR analysis of the Phosphatase I mRNA expression in diseased (i.e. atherosclerotic) ventricular tissue gave a strong positive signal after staining. However, no such signal was observed following RNAse treatment of the tissue.

Experimental

The following examples serve to illustrate certain preferred embodiments and aspects of the present invention and are not to be construed as limiting the scope thereof.

In the experimental disclosure which follows, the following abbreviations apply: DAPI (4′,6-diamiidino-2-phenylindole); eq (equivalents); FITC (fluorescein isothiocyanate); M (Molar); μM (micromolar); N (Normal); mol (moles); mmol (millimoles); μmol (micromoles); nmol (nanomoles); g (grams); mg (milligrams); μg (micrograms); ng (nanogram); L (liters); ml (milliliters); μl (microliters); cm (centimeters); mm (millimeters); μm (micrometers); nm (nanometers); ° C. (degrees Centigrade); PBS (phosphate buffered saline); SDS (sodium dodecylsulfate); SSC (sodium salt citrate); Tris-HCl (tris[Hydroxymethyl]aminomethane-hydrochloride); rpm (revolutions per minute); YOYO-1 (YO-YO-1 iodide); LoM (low-melting point); EDTA (ethylenediaminetetracetic acid); DCTP (2′-deoxycytidinine 5′-triphosphate); dUTP (2′-deoxyuridine 5′-triphosphate); ATCC (American Type Culture Collection, Rockville, Md.); Roche Molecular (Roche Molecular Biochemicals, Indianapolis, Ind.); Chroma (Chroma Technology, Brattleboro, Vt.); DAKO (DAKOPATTS, Carpintera, Calif.); Difco (Difco Laboratories, Detroit, Mich.); Fisher (Fisher Scientific, Pittsburg, Pa.); Gibco-BRL (Gibco-BRL Life Technologies, Inc., Rockville, Md.); ICN (ICN Biochemicals, Costa Mesa, Calif.); Molecular Probes (Molecular Probes, Eugene, Oreg.); NEB (New England Biolabs, Beverly, Mass.); Sigma (Sigma Chemical Co., St. Louis, Mo.); US Biochemical (US Biochemical Corp., Cleveland, Ohio); Vector (Vector Laboratories, Burlingame, Calif.); Zeiss (Carl Zeiss, Inc., Thornwood, N.Y.); Promega (Promega Corp., Madison, Wis.); AB (Applied Biosystems, Foster City, Calif.)

Unless otherwise noted, all restriction enzymes were obtained from Boehringer Mannheim and restriction digests were performed according to the manufacturer's instructions.

The following reagents were obtained from Vector: Texas red anti-sheep IgG (H&L); fluorescein anti-mouse IgG (H&L); AMCA avidin D; biotinylated anti-avidin D; and fluorescein avidin DCS. Anti-digoxigenin-rhodamine, Fab fragments and Anti-digoxigenin-fluorescein, Fab fragments were obtained from Roche Molecular. Avidin-CY5 was obtained from Biological Detection Systems (Pittsburgh, Pa.). Avidin Neutralite Cascade Blue was obtained from Molecular Probes.

### EXAMPLE 1

Preparation of Paraffin-Embedded Tissue

This example provides a standard protocol by which cardiovascular tissues may be embedded in paraffin. Paraffin embedding is a process in which the tissue specimen is fixed to preserve its cellular structures, and blocked out and embedded in paraffin to stabilize it for long-term storage and easy sectioning. Upon processing, the tissue may be sectioned, mounted, and subjected to Laser Capture Microdissection, DNA and/or RNA extraction, or analysis by any means contemplated by the invention. Briefly, cardiovascular tissue specimens are arrayed in a paraffin embedding block as described above in Part II. Said tissues are fixed at separate workstations by sequential incubation in ethanol and xylene (concentrations and incubation times are as provided in the table directly below) at 40° C. The fixed cardiovascular tissues are then treated with paraffin in order to embed them in the embedding block (incubation times and temperatures are as provided in the table directly below).

| Workstation | Solution | Concentration | Time (min) | Temp.(° C.) |
|---|---|---|---|---|
| 1. | Ethanol | 70% | 0:30 | 40 |
| 2. | Ethanol | 80% | 0:30 | 40 |
| 3. | Ethanol | 95% | 0:45 | 40 |
| 4. | Ethanol | 95% | 0:45 | 40 |
| 5. | Ethanol | 100% | 0:45 | 40 |
| 6. | Ethanol | 100% | 0:45 | 40 |
| 7. | Ethanol | 100% | 0:45 | 40 |
| 8. | Xylenes | 100% | 0:45 | 40 |
| 9. | Xylenes | 100% | 0:45 | 40 |
| 10. | Paraffin | | 0:30 | 58 |
| 11. | Paraffin | | 0:30 | 58 |

After processing as described above, the specimen is now embedded in paraffin. Sections, or ribbons, of the paraffin embedded cardiovascular tissue are cut on a clean microtome with a clean blade. The sectioning of paraffin blocks at 5.5 microns in thickness is optimal for LCM, but the thickness should be dependent on the tissue or cell (nuclei) diameter that is being processed. The paraffin ribbons are floated in 43-44° C. deionized water (no adhesives), and then subsequently mounted upon plain (uncoated) glass slides (e.g. a conventional microscope slide).

### EXAMPLE 2

### Rematoxylin and Eosin Staining

This example provides a protocol for the staining of cardiovascular tissues for immunological or histological analysis as contemplated by the present invention. Fresh staining solutions are prepared and staining vessels are arranged to facilitate moving through the staining procedure in a timely manner. This should ensure that the tissue does not stand at room temperature for any period of time until it is completely dry (after the last Xylene treatment). The cardiovascular tissue samples are sectioned and mounted before proceeding. The paraffin is removed from the cardiovascular tissue paraffin sections prior to continuing (as described below in Example 3). The de-paraffinzed tissue microarray is stained by performing the following sequential incubations at room temperature: 1) 70% Ethanol for 30 seconds; 2) dH₂0 for 30 seconds; 3) Hematoxylin for 30 seconds; 4) dH₂0 for 30 seconds; and 5) Bluing Reagent for 30 seconds. Then, the microarray is rinsed sequentially in 70% and 95% Ethanol, each for 30 seconds, and stained with Eosin for 30 seconds. The microarray is sequentially rinsed in 70%, 95%, and 100% Ethanol, each for 30 seconds. The microarray is fixed in Xylene for 5 minutes, with an optional additional Xylene treatment for 5 minutes. The microarray is air dried for 20 minutes in a fume hood or vacuum desiccator. The tissue sections are now ready for LCM as described below. (See Example 5). The samples are stored in a desiccator when not in use.

### EXAMPLE 3

De-Paraffinization of Cardiovascular Tissue Sections

This example provides a protocol by which paraffin-embedded tissues contemplated by the invention may be de-paraffinized. Paraffin is removed from cardiovascular tissue microarrays (prepared as described above) by the following method. Cardiovascular tissue sections that have been mounted onto glass slides to form a microarray, and air-dried overnight, are deparaffinized by dipping the slide containing the tissue section into Copplin Jars (or other solvent containers) containing the following solutions for the specified times:

| Solution | Incubation Time |
|---|---|
| Xylene | 5 minutes |
| Xylene | 5 minutes |
| 100% Ethanol | 30 seconds |
| 95% Ethanol | 30 seconds |
| 70% Ethanol | 30 seconds |
| Distilled Water | 30 seconds |

After sequential incubation as directed above, the de-paraffinized tissue (i.e. on the microarray) is ready for use in the detection of biomarkers associated with Cardiovascular Disease as contemplated by the present invention (e.g. by histological analysis, immunological analysis, and/or nucleic acid hybridization analysis).

### EXAMPLE 4

Immunohistochemical Staining (IHC) for Laser Capture Microdissection

This example provides a protocol for a method of immunological analysis as contemplated by the present invention. For optimal Laser Capture Microdissection (LCM) from IHC samples, it is necessary to minimize the amount of time the samples are incubated in an aqueous environment. Fewer and shorter incubation steps are recommended. For the best results, use charged or poly-L-lysine coated slides for mounting cardiovascular tissue sections. Tissues may not adhere through the entire IHC process on a plain glass slide. Prior to staining, the concentration of antibody to be used is determined through titration. For this protocol, double the normal antibody concentration is used. For RNA work, it is recommended to add 1 unit/μl of RNAse inhibitor to the antibody solutions and to use RNAse-free glassware and reagents. For paraffin embedded tissue sections, the sections are de-paraffinized first. For frozen tissue sections, the sections are fixed in 70% Ethanol for 30 seconds, or Acetone (4° C.) for 4 minutes. If mounted sections were removed from the −80° C. freezer, allow 30 seconds for the condensation to disappear prior to fixing. Then, the following steps are performed.

The tissues are rehydrated in phosphate buffered saline (PBS) for 30 seconds. The tissue microarray is blocked with normal serum (1:10 in PBS) from the same animal species that the secondary antibody was raised in. (This step is optional depending on the amount of background staining that may appear). The excess serum is blotted-off the microarray by tapping the edge of the slide onto a clean paper towel. The primary antibody (e.g. goat anti-human PAI-1 IgG) is applied for 10 minutes at room temperature, and the microarray is rinsed in PBS for 10 seconds, 3 times. Then, the secondary antibody (e.g. anti-goat IgG) is applied for 10 minutes at room temperature. Note, that the secondary antibody may either be conjugated to an enzyme or a fluorescent moiety to aid in detection. A small amount of fluorophore (e.g. TRITC) would be added to develop a fluorescently labeled secondary antibody, whereas, with an enzyme conjugated antibody, horseradish peroxidase (HRP) or alkaline phosphatase (AP) is added. The tissue microarray is rinsed in PBS. If using an enzyme conjugated secondary antibody, the reaction is developed under the microscope. Substrates that do not result in a black colored product, such as DAB for HRP and Vector Red for AP, are used. The tissue microarray is rinsed with dH₂0. The tissues are dehydrated by sequential incubation in: 1) 75% Ethanol for 30 seconds; 2) 95% Ethanol for 30 seconds; 3) 100% Ethanol for 30 seconds; and 4) Xylene for 5 minutes. Finally, the microarray is dried in a fiune hood for 20-30 minutes. The samples are now ready for LCM as described below. (See Example 5). Samples are stored in a desiccator and protected from light, when not being used.

### EXAMPLE 5

Laser Capture Microdissection (LCM)

LCM allows precise identification, dissection, and harvesting of pure cell or tissue populations that are more representative of the disease process in vivo than cells in culture that are distorted by conditions and selection pressures. Ohyama et al., “Laser Capture Microdissection-Generated Target Sample for High-Density Oligonucleotide Array Hybridization,” Biotechniques, 29: 530-36 (2000). Through the use of LCM, desirable cardiovascular tissues placed on microarrays and stained as described above in Example 3 or 4 may be selectively harvested for nucleic acid isolation. Such nucleic acids are extracted from the cardiovascular tissue sections as described below (See Examples 6 & 7) and utilized downstream in nucleic acid hybridization analysis.

In one embodiment, cardiovascular tissue sections are prepared as described below (See Example 8) and mounted on plain microscope slides. A Prep Strip is applied to flatten the tissue. Any loose tissue debris is removed prior to LCM. A piece of transfer film, CapSure HS (Arcturus Engineering, Mountainview, Calif.: Cat.#TFHS-SP), is placed onto the tissue in the area of interest. The CapSure HS keeps the tissue-transfer film out of contact with the tissue. The low power infrared laser is pulsed over the tissues and cells of interest (as differentiated by their prior staining) thereby activating the transfer film which then expands down onto contact with the tissue. The desired tissues or cells now adhere to the CapSure HS transfer film. The CapSure HS transfer film carrier with the desired cells is lifted off the slide, leaving the remaining tissue intact. The transfer film containing the desired cardiovascular tissues is subsequently processed by DNA and/or RNA extraction (See Examples 6 & 7) to yield nucleic acids for downstream analysis.

### EXAMPLE 6

DNA Extraction

The following example provides a protocol useful in the extraction of deoxyribonucleic acids from tissues as contemplated by the present invention. The following extraction method is used for Polymerase Chain Reaction (PCR), measuring loss of heterozygosity (LOH), dideoxy fingerprinting (DDF), clonality analysis (chromosome X inactivation), and direct sequencing of PCR products for single base mutational analysis. Extractions are typically performed on 500-1000 laser captured cells from the LCM procedure as described above. (See Example 5).

Pipet 50 μl of proteinase K digestion buffer (1 mg/ml Proteinase K, 1% Tween 20* in TE Buffer pH 8.0) into a microfuge tube. Place the tube up-right in a humidified incubator at 37-42° C. for 5 minutes to pre-heat. Place the CapSure Transfer Film cap (ie. the transfer film containing the desired cardiovascular tissues or cells) onto the microfuge tube with the insertion tool. Invert the microfuge tube and flick down the digestion buffer until all the fluid is in contact with the surface of the cap containing the sample. Incubate overnight in the humidified incubator at 37-42° C. Centrifuge the microfuge tube for 5 minutes at a maximum of 6400 rpm (2000×g) to collect the fluid and DNA. Remove the transfer film cap and visually inspect under the microscope to verify that all cells have been digested (no cellular material remains on the transfer film areas of capture). Inactivate the proteinase K by heating the microfuge tube at 95° C. for 10 minutes. The sample is now ready to go directly into PCR amplifications, or into other applications.

### EXAMPLE 7

RNA Extraction

The following example provides a protocol useful in the extraction of deoxyribonucleic acids from tissues as contemplated by the present invention. The following extraction method is used for the purification of ribonucleic acids for use in nucleic acid hybridization analysis (e.g. in situ RT-PCR) as contemplated by the present invention. Extractions are typically performed on 500-1000 laser captured cells from the LCM procedure as described above. (See Example 5).

This RNA extraction utilizes an RNA denaturing buffer (GITC) that is comprised of 5.25M GITC (guanidinium isothiocyanate), 5 mM Tris-Cl, pH 6.4, 20 mM EDTA, and 1% Triton X-100. Place the CapSure Transfer Film cap (i.e. the transfer film containing the desired cardiovascular tissues or cells) onto the microfuge tube (with the insertion tool) containing 200 μl RNA denaturing buffer (GITC) and 1.6 μl 0.1M β-mercaptoethanol. Invert several times over the course of 2 minutes to digest the tissue off of cap. Remove the solution from the microfuge tube and place it in a clean 1.5 ml tube. Sequentially add 20 μl (0.1× volume) 2M sodium acetate (pH 4.0), 220 μl (1X volume) water saturated phenol (bottom layer), and 60 μl (0.3× volume) chloroform-isoamyl alcohol (49:1) to the microfuge tube and vortex vigorously. Put the microfuge tube on wet ice for 15 minutes. Centrifuge the microfuge tube for 30 minutes at 4° C. to separate the aqueous and organic phases. Transfer the upper (aqueous) layer to a new tube. Add 1-2 μl glycogen (10 mg/ml) and 200 μl cold isopropanol to the microfuge tube. Put the microfuge tube in a −80° C. freezer for at least 30 minutes, or overnight. Centrifuge the microfuge tube for 30 minutes at 4° C. with its cap hinges pointing outward so that the location of the pellet can be better predicted. Remove the majority of the supernatant with a 1000 μl pipet tip, and then switch to a smaller pipet to remove the rest of the supernatant. Wash the pellet with 400 μl cold 70% Ethanol and spin the microfuge tube for 5 minutes at 4° C. Remove the supernatant as explained above. All of the supernatant should be removed at this point. Let the pellet air dry on ice to remove any residual ethanol. Pellets can be stored at −80° C. until use. When ready for use, pellets may be resuspend DEPC-treated water.

If complete removal of DNA from the RNA sample is desired, a DNAse treatment, followed by re-extraction of the RNA, may be performed as an optional step the above protocol. Briefly, to an RNA pellet, add 15 μl DEPC water, 1 μl of 20 units/μl RNAase inhibitor (Perkin Elmer), 2μl 10× DNAase buffer (Genhunter), and 2 μl 10 units/μl DNAase (20 units total). Incubate the RNA pellet at 37° C. for 2 hours in order to digest any remaining DNA. After digestion, add 2 μl 2M NaOAc (pH 4.0), 22 μl Phenol (water-saturated), and 6 μl Chloroform-isoamyl alcohol (49:1) to the microfuge tube in order to re-extract the RNA. Place the microfuge tube on ice for 15 minutes, and centrifuge for 10 minutes at 4° C. Transfer the upper layer (aqueous phase) to a new tube. Continue with RNA extraction as detailed above beginning with the addition of 1-2 μl glycogen (10 mg/ml) and 200 μl cold isopropanol to the microfuge tube.

### EXAMPLE 8

Preparation of Frozen Tissue for LCM

This example provides a protocol by which tissues contemplated by the present invention may be processed for the detection of biomarkers associated with cardiovascular disease by histological, immunological, or nucleic acid hybridization analysis. Frozen embedding is another way to preserve specimens and stabilize them for long-term storage and sectioning. Tissue is embedded in a viscous compound, such as O.C.T. (Tissue-Tek) and deep-frozen at dry ice, or at a lower temperature (e.g. freezing with liquid nitrogen). This method has the benefits of faster processing and excellent molecular recovery. Up to 800 base pairs for both RNA and DNA have been recovered from O.C.T.-embedded tissue, and up to 2 kilobases in cDNA library smears.

Embedding

1. Place an empty, labeled cryomold on dry ice for 1 min. It should remain on dry ice during the entire embedding procedure.

2. Cover the bottom of the cryomold with embedding medium (i. e., O.C.T.)

3. Place the frozen tissue against the bottom. To facilitate cutting, the tissue should be relatively small (1 cm) and the desired cutting surface should be flush against the bottom.

4. Fill the cryomold with embedding medium. Cover the dry ice container and allow the O.C.T. to harden (it will turn white when frozen).

5. Wrap the block in foil and keep at −80° C. until cutting.

Cutting

1. Remove the block from the cryomold (if not already done) and attach it to the chuck in the cryostat with O.C.T. The cutting surface should be as parallel as possible.,

2. Allow the block to equilibrate to the cryostat temperature (−20° C.) for about 15 minutes. If the block is too cold during cutting, this time may need to be extended.

3. Cut 10 μm (or thinner) sections onto plain uncoated, charged or silanated glass slides. If necessary, the sections may be cut thinner or thicker.

4. Keep the slides in the cryostat or on dry ice if LCM is to be performed that day. Alternatively, they may be stored in paper slide boxes at −80° C. until needed.

Transfer

For optimal transfer of frozen tissue sections, it is best to keep sections <10 μm thick. Thicker sections are more difficult to visualize. If there are folds in the tissue, the cap may not make direct contact with the entire surface at that area. Therefore, it is advisable to inspect the tissue before placing down the cap. If any tissue seems to be mounded or folded, it is best not to place the cap over that area.

The tissue section must be dry and not coverslipped for effective LCM transfer. The staining appears darker and more granular due to light scattered from the irregular air-tissue interface. The tissue where the polymer melts and bonds after laser activation appears lighter and resembles a coverslipped slide due to the replacement of the air in the tissue with the polymer. This phenomenon is called index-matching or polymer wetting.

Poor transfers may result if the slide is not fully dehydrated (i.e. the 100% ethanol becomes hydrated after repeated use). The final xylene rinse facilitates the efficiency of transfer with LCM. While other staining protocols can be used, the slides should be dehydrated in a final xylene step.

### EXAMPLE 9

In Situ Reverse Transcriptase Polymerase Chain Reaction (IS RT-PCR)

In this example, a method for the in situ RT-PCR of human total RNA to detect the presence, absence, or variation in the expression level of the human PAI-1 gene is described as follows.

a. Section Preparation and IS RT-PCR

Sections of human cardiovascular tissue (e.g. 5.5 μm thick) are mounted on uncoated slides and dried at room temperature as described above in Example 1. The slides are then rinsed three times for 10 minutes in xylenes, followed by dehydration twice for 10 minutes each time in 100% ethanol. The sections are mildly digested by Proteinase K (0.1 mg/ml in 50 mM Tris-HCI pH 7.6, 5 mM EDTA; Sigma Chemical, St. Louis, Mo.) for 12 minutes at room temperature. The digestion is stopped by several washes in Tris-buffered saline (TBS).

A volume of 25 μl of a reverse transcriptase solution containing 1×PCR buffer II, 5 mM MgCl₂, 10 mM dNTP mixture, 2.5 μM Oligo d(T)₁₆, 20 units RNase inhibitor, and 100 units MuMLV reverse transcriptase (Perkin-Elmer, Foster City, Calif.) is applied to each section in Frame-Seal Incubation Chambers (MJ Research, Watertown, Mass.) on slides. The sealed slides are placed on a heat block of the PCR Thermal Cycler (GeneAmp In Situ PCR System 1000, Perkin-Elmer) and incubated according to a temperature program as follows: 30° C. for 30 minutes; 42° C. for one hour followed by 5° C. for 10 minutes. After incubation, the solution in the incubation chambers is carefully removed by pipetting, and the section is briefly washed in PBS buffer.

PCR amplification of the cDNA generated above is performed using a GeneAmp DNA amplification reagent kit (Perkin-Elmer, Norwalk, Conn.) with human PAI-1 specific primers (i.e. primers 1 & 2), and with primers specific to GAPDH (5′-CCC TCC GAC GCC TGC TT-3′ and 5′-ATC ATC AGC AAT GCC TCC TG-3′) as an internal control. Of the resulting cDNA, 2 μl is mixed in a PCR reaction containing 1.5 μl of 10 mM dNTP, 2 μl 10×PCR buffer, 0.6 μl 50 mM MgCl₂, 0.2 μl Taq DNA Polymerase, 10 pmol of primer 1 (5′-GGA ACA AGG ATG AGA TCA GC-3′), 10 pmol of primer 2 (5′-CTG GCC GTT GAA GTA GAG G-3′), 10 pmol of each internal control-specific primers, and 10.8 μl of sterile water. The slide is sealed in an incubation chamber, placed in the PCR Thermal Cycler, and subjected to 26 cycles of amplification comprising denaturation at 94° C. for 1 minute, annealing at 60° C. for 1 minute, polymerization at 72° C. for 3 minutes, and a separate and final extension cycle at 72° C. for 7 minutes. The primary PCR product is labeled during a second PCR by including a single cycle of denaturation, annealing, and extension (94° C. for 1 minute, 60° C. for 90 seconds, and 72° C. for 90 seconds) in 25 μl of the PCR mixture containing 0.2 mM of DATP, dCTP and dGTP, 0.13 mM of dTTP, and 0.07 mM of digoxigenin (DIG)-II-dUTP (Roche Molecular Biochemicals, Indianapolis, Ind.).

b. Digoxigenin Detection

After labeling the PCR products, the slides are washed twice in Tris-NaCl (0.1 M Tris, pH 7.5, 0.15 M NaCl) for 10 minutes, and immersed in blocking buffer (Roche Molecular Biochemicals) for 15 minutes. The detection of the incorporated DIG-labeled dUTP (and thus, the presence, absence, or variation in the expression of, the PAI-1 gene) is performed with a 100 μl of highly specific anti-DIG antibody conjugated with alkaline phosphatase (Roche Molecular Biochemicals) solution, which is diluted 1:500 in blocking buffer, and then incubated with the slides for 30 minutes at room temperature. For visualization of the PAI-1-specific RT-PCR product, the slides are incubated in 15 ml of freshly prepared nitroblue tetrazolium/5-bromo-4-chloro-3-indolylphosphate (Roche Molecular Biochemicals) for 15-30 minutes.

### EXAMPLE 10

Specific Primers Utilized and Genes Amplified by the Present Invention

This example provides a description of some of the genes that may be amplified in performing nucleic acid hybridization analysis (e.g. in situ RT-PCR) as contemplated by the present invention.

a. Human Cytoskeletal Beta-Actin (High Number Copies)

Human cytoskeletal β-actin is a cytoskeletal protein that is expressed in high abundance (0.3-1%) in the following tissues: adipose tissue; adrenal gland; bone; brain; breast; colon; embryo; endothelial cells; eye; gall bladder; greater ementum; heart; kidney; liver; lung; lymphoid tissue; ovary; pancreas; placenta; prostate; skeletal muscle; skin; smooths muscle; spleen; synovial membrane; testis; thymus gland; thyroid gland; uterus; white blood cells. The size of β-actin RNA is 1761 nucleotides in length and its nucleotide sequence can be found in GenBank. (Accession Nos. X00351, J00074, M10278, and M10277). Primers for in situ RT-PCR were designed based on said nucleotide sequence and have the following sequences:

1) sense 5′-GGGAAATCGTGCGTGACATTAAGGA-3′ (based on nucleotide numbers 658-680); and

2) anti-sense 5′-TGTGTTGGCGTACAGGTCTTTGC-3′ (based on nucleotide numbers 932-911).

Said primers generate a PCR product size of 275 nucleotides in length for RNA, or 370 nucleotides in lenght for gDNA. (See Ponte et al., “Evolutionary conservation in the untranslated regions of actin mRNAs: DNA sequence of a human beta-actin cDNA,” Nucleic Acid Res, 12: 1687-1696 (1984)).

b. Glycerlaldehyde 3′-Phosphate Dehydrogenase (GAPDH)

GAPDH is a cytoskeletal protein that is expressed in the following tissues: adipose tissue; adrenal gland; bone; brain; breast; colon; embryo; endothelial cells, epididymis, eye; gall bladder; heart; kidney; liver; lung; lymphoid tissue; ovary; placenta; platelet; prostate; skeletal muscle; skin; smooth muscle; spleen; synovial membrane; thymus gland; thyroid gland; uterus; white blood cells; testis. The size of GAPDH RNA is 1268 nucleotides in length and its nucleotide sequence can be found in GenBank (Accession Nos. M33197 and J04038). Primers for in situ RT-PCR were designed based on said nucleotide sequence and have the following sequences:

| | |
|---|---|
| 1) sense | 5′-CCCTCCGACGCCTGCTT-3′; |
| and | |
| 2) anti-sense | 5′-ATCATCAGCAATGCCTCCTG-3′. |

Said primers generate a PCR product size of 352 nucleotides in length for RNA, or 556 nucleotides in lenght for gDNA.

C. Human Protein Phosphatase 1 (Low Abundance Genes)

The catalytic subunit of the Human protein phosphatase 1 (HPP-1) is involved in the dephosphorylation of proteins and intracellular signaling. HPP-1 is expressed in low abundance (0.02%) in the following tissues: adrenal gland; embryo; heart; placenta; prostate; skeletal muscle; testis; and uterus. The size of HPP-1 RNA is 1374 nucleotides in length and its nucleotide sequence can be found in GenBank (Accession No. X70848). Primers for in situ RT-PCR were designed based on said nucleotide sequence and have the following sequences:

1) 5′-AAGTACCCCGAGAACTTCTTCCTG-3′ (based on nucleotide nos. 346-387); and

2) 5′-TGAGGTCCAAGTCGTGCTTGTG-3′ (based on nucleotide nos. 757-736).

Said primers generate a PCR product size of 394 nucleotides in length for RNA. (See Song et al., “Cloning and characterization of a human protein phosphatase 1-encoding cDNA,” Gene 129: 291-295 (1993)).

d. Plasminogen Activator Inhibitor 1 (PAI-1)

PAI-1 is part of the class of arterial serpins that regulate steps in the thrombotic and thrombolytic cascades. PAI-1 is produced by cells in the vessel wall and acts to inhibit plasminogen activators such as TPA, and is expressed in the following tissues: adipose, aorta, bone, brain, breast, colon, esophagus, foreskin, gall bladder, heart, kidney, liver, lymph, ovary, pancreas, placenta, prostate, skin, stomach, thymus, umbilical vein, uterus, adrenal gland, connective tissue, genitourinary tract, and lung. The size of PAI-1 RNA is 2876 nucleotides in length and its nucleotide sequence can be found in GenBank (Accession No. M16006.1). Primers for in situ RT-PCR were designed based on said nucleotide sequence and have the following sequences:

| | |
|---|---|
| 1) sense | 5′-TCCACAAATCAGACGGCAG-3′; |
| and | |
| 2) anti-sense | 5′-CATAAGGGGCAGCAATGAAC-3′ |

Said primers generate a PCR product size of 159 nucleotides in length for RNA.

e. Human Cytoskeletal Gamma Actin (Medium Number Copies)

Human cytoskeletal y-actin is a cytoskeletal protein that is expressed in medium abundance (0.3-1%) in the following tissues: adipose tissue; adrenal gland; bone; brain; breast; colon; embryo; endothelial cells; epididymis; eye; gall bladder; heart; kidney; liver; lung; lymphoid tissue; ovary; placenta; platelet; prostate; skeletal muscle; skin; smooth muscle; spleen; synovial membrane; thymus gland; thyroid gland; uterus; white blood cells; and testis. The size of γ-actin RNA is 1761 nucleotides in length and its nucleotide sequence can be found in GenBank (Accession Nos. X00351, J00074, M10278, and M10277). Primers for in situ RT-PCR were designed based on said nucleotide sequence and have the following sequences:

| | |
|---|---|
| 1) sense | 5′-TGTGTTGGCGTACAGGTCTTTGC-3′; |
| and | |
| 2) antisense | 5′-ATCATCAGCAATGCCTCCTG-3′. |

Said primers generate a PCR product size of 275 nucleotides in length for RNA, or 370 nucleotides in length for gDNA.

What is claimed is:

1. A device having a substantially flat surface, wherein a plurality of human cardiovascular tissue samples form at least one human donor are arrayed on said surface, wherein said plurality comprises a first sample from a first disease state and a second tissue sample from a second disease state.

2. The device of claim 1, wherein the second disease state is a later disease state than said first disease state.

3. The device of claim 1, wherein said plurality of human cardiovascular tissue samples further comprise non-diseased cardiovascular tissue controls.

4. The device of claim 1, wherein at least a portion of said human cardiovascular tissue samples comprise cadaveric donor specimens.

5. The device of claim 1, wherein said human cardiovascular tissue samples are derived from more than one human source.

6. The device of claim 1, wherein said human cardiovascular tissue samples are selected from the group consisting of human venous and arterial vessels, ventricles, and auricles.

7. A device having a substantially flat surface, wherein a plurality of human cardiovascular tissue samples from at least one human donor are arrayed on said surface, wherein said plurality comprises a first sample of a first tissue type and a second tissue sample of a second tissue type.

8. The device of claim 7, wherein said plurality of human cardiovascular tissue samples further comprise non-diseased cardiovascular tissue controls.

9. The device of claim 7, wherein at least a portion of said human cardiovascular tissue samples comprise cadaveric donor specimens.

10. The device of claim 7, wherein said human cardiovascular tissue samples are derived from more than one human source.

11. The device of claim 7, wherein said human tissue samples are selected from the group consisting of human venous and arterial vessels, ventricles, and auricles.

12. A device having a substantially flat surface, wherein a plurality of tissue samples from at least one human donor are arrayed on said surface, wherein said plurality comprises a first group of tissue samples and a second group of tissue samples, wherein said first group comprises cardiovascular tissue samples and said second group comprises non-cardiovascular tissue.

13. The device of claim 12, wherein at least a portion of said cardiovascular tissue samples of said first group are from diseased tissue.

14. The device of claim 13, wherein at least a portion of said diseased cardiovascular tissue samples are from at least two different cardiovascular disease states.

15. The device of claim 14, wherein the two different disease states are temporally different disease states.

16. The device of claim 13, wherein at least a portion of said diseased cardiovascular tissue samples are from at least two different cardiovascular tissue types.

17. The device of claim 12, wherein at least a portion of said non-cardiovascular tissue samples of said second group are from healthy tissue.

18. The device of claim 12, wherein at least a portion of said non-cardiovascular tissue samples are from at least two different non-cardiovascular tissue types.

19. A method comprising:
a) providing the device of claim 1; and
b) treating said plurality of human cardiovascular tissue samples under conditions such that a biomarker associated with cardiovascular disease is detected.

20. The method of claim 19, wherein said treating comprises histological analysis.

21. The method of claim 20, wherein said histological analysis comprises immunohistochemistry.

22. The method of claim 19, wherein said treating comprises nucleic acid hybridization analysis.

23. The method of claim 19, wherein said treating comprises immunological analysis.

24. A device having a substantially flat surface, wherein a plurality of human cardiovascular tissue samples from at least one donor are arrayed on said surface, wherein said plurality comprises a first sample from a first region of the heart and a second sample from a second region of the heart.

25. The device of claim 24, wherein said first region is represented by section A-1 of FIG. 8.

26. A device having a substantially flat surface, wherein a plurality of human cardiovascular tissue samples from at least one donor are arrayed on said surface, wherein said plurality comprises tissue from a donor suffering from a cardiovascular disease selected from the group consisting of stable angina, unstable angina, non-Q-wave myocardial infarction, and Q-wave myocardial infarction. In another embodiment, said plurality further comprises non-diseased cardiovascular tissues.

27. A device having a substantially flat surface, wherein a plurality of human cardiovascular tissue samples from at least one donor are arrayed on said surface, wherein said plurality comprises a first sample from a first region of the heart and a second sample from a second region of the heart, and wherein said plurality comprises tissue from a donor suffering from a cardiovascular disease selected from the group consisting of stable angina, unstable angina, non-Q-wave myocardial infarction, and Q-wave myocardial infarction.
